(19) United States
(12) Patent Application Publication
(10) Pub. No.: US 2023/0026406 A1
(43) Pub. Date: Jan. 26, 2023

(54) THERAPEUTIC FRACTIONS AND PROTEINS FROM ASTHMA-PROTECTIVE FARM DUST

(71) Applicant: ARIZONA BOARD OF REGENTS ON BEHALF OF THE UNIVERSITY OF ARIZONA, Tucson, AZ (US)

(72) Inventors: Fernando D. Martinez, Tucson, AZ (US); Donata Vercelli, Tucson, AZ (US); Shane A. Snyder, Tucson, AZ (US); Erika R. VonMutius, Tucson, AZ (US); Vadim Pivniouk, Tucson, AZ (US); Mauricius Marques dos Santos, Tucson, AZ (US)

(21) Appl. No.: 17/817,178

(22) Filed: Aug. 3, 2022

Related U.S. Application Data
(63) Continuation-in-part of application No. PCT/US2021/016918, filed on Feb. 5, 2021.
Application No. PCT/US2021/016918 is a continuation-in-part of application No. 17/413,468, filed on Jun. 11, 2021.
(60) Provisional application No. 62/971,633, filed on Feb. 7, 2020.

(30) Foreign Application Priority Data
Dec. 14, 2018 (LU) LU101064

(51) Int. Cl. A61K 39/35; A61P 37/08; A61P 11/06; A61K 31/202

(52) U.S. Cl. CPC A61K 39/35; A61P 37/08; A61P 11/06; A61K 31/202

(57) ABSTRACT

The present invention relates to the asthma-protective effects of farm dust, specifically to a composition comprising barn dust extract including isolated fractions of an Amish barn dust extract comprising different bioactive components that have an ability to protect against asthma. In particular, the present invention describes a barn dust composition with asthma-protective properties, the barn dust composition comprising one or more bioactive fractions extracted from barn dust, said one or more bioactive fractions comprising one or more proteins and one or more fatty acids. The one or more bioactive fractions of the barn dust compositions may comprise molecular weights of 30-100 kDa, particularly 28-64 kDA, that include eight (8) target proteins, which have potential to proactively prevent the induction of asthma and to treat current cases of asthma. The present invention also relates to an in vitro method for screening allergic compounds.

### CROSS-REFERENCES TO RELATED APPLICATIONS

This application is a continuation-in-part and claims benefit of PCT Application No. PCT/US2021/016918 filed Feb. 5, 2021, which claims benefit of U.S. Provisional Application No. 62/971,633 filed Feb. 7, 2020, the specifications of which are incorporated herein in their entirety by reference.

This application is a continuation-in-part and claims benefit of U.S. patent application Ser. No. 17/413,468 filed Jun. 11, 2021, which is a 371 application of PCT Application No. PCT/EP2019/085016 filed Dec. 13, 2019, which claims benefit of LU101064 filed Dec. 14, 2018, the specifications of which are incorporated herein in their entirety by reference.

### FIELD OF THE INVENTION

The present invention relates to the asthma-protective effects of farm dust, specifically to a composition comprising barn dust extract including isolated fractions of an Amish barn dust extract comprising different bioactive components that have an ability to protect against asthma. In particular, the present invention describes bioactive fractions of Amish farm dust comprising molecular weights of 30-100 kDa, particularly 28-64 kDA, that include eight (8) target proteins, which have potential to proactively prevent the induction of asthma and to treat current cases of asthma. The eight (8) proteins comprise odorant-binding protein (OBP), allergen Bos d2, interferon gamma, provicilin, vicilin (14 kDa component), beta-conglycinin, sarcoplasmic calcium-binding protein, and MATH domain At3g58400. The barn dust extract is useful in the prevention or treatment of allergy-related diseases. The present invention also relates to an in vitro method for screening allergic compounds.

### BACKGROUND OF THE INVENTION

Currently, over 300 million people worldwide suffer from asthma, a chronic disease in which the bronchial tubes of the lungs become inflamed causing restricted airflow. This inflammation of the airways makes it difficult to breathe. Symptoms of asthma include coughing, wheezing, shortness of breath, and tightening of the chest. The exact cause of asthma is unknown but both genetic and environmental factors have been shown to play a role in the development of asthma. For example, the likelihood of having asthma is significantly increased if your parents had asthma. Allergy is also highly prevalent in the Western world, and rapidly on the rise in highly populated countries like China. Costs of treating these diseases are extremely high; recent prescription claims data show that for example, approximately two-thirds of patients with allergic rhinitis receive treatment with one or more medications from the non-sedating antihistamines and nasal glucocorticoids classes, with expenditures exceeding $3.0 billion for prescription antihistamines alone. Novel strategies that effectively prevent and treat asthma and allergies are therefore urgently needed.

There is currently no cure for asthma but a large majority of children “grow-out” of their asthma. Prevention and long-term control are used to prevent asthma attacks. Typically, allergy medicine and limiting physical exertion are used to prevent triggers of attacks. Long-term control medications, such as inhaled corticosteroids, are anti-inflammatory drugs that are usually taken once daily and have relatively low risk of side effects but take days to weeks before proving fully beneficial and do not change the natural history of the disease. Leukotriene modifiers that can be used as oral medications offer 24 hours of relief with rare side effects including psychological reactions. Combination inhalers use a combination of a corticosteroid and a long-acting beta agonist but may increase risk of having a severe asthma attack. Theophylline can be taken as a daily pill that relaxes the muscles around the airways, but its use has been declining. Quick-relief medications include short acting beta agonists and quick relief bronchodilators (e.g., ipratropium) and are predominately used for chronic bronchitis rather than asthma. Oral and IV corticosteroids relieve airway inflammation caused by severe asthma.

There has been movement in trying to treat asthma using immunotherapy techniques by exposure to allergens. There are two main types of immunotherapy: 1) sublingual immunotherapy (SLIT), allergens are administered under the tongue, SLIT is currently only FDA approved for ragweed and grass pollen allergy treatment; and 2) subcutaneous immunotherapy (SCIT), allergens are administered under the skin. However, documentation around SLIT and SCIT products has been conflicting and often unreliable. Better clinical trials are needed to accurately show efficacy of this therapy for the treatment of asthma.

Relevant prior art patent applications describe 1) anti-inflammatory peptides, e.g., a homolog of provicilin with similar sequence identity, and their uses to decrease inflammation including inflammation caused by asthma (EP3118215A1); 2) allergens (some of the allergens suggested to use are those presented within the eight (8) proteins of the present invention) to treat allergies before a subject has developed any symptoms of allergies (US20170087231A1); and 3) a process for the preparation of an antiallergenic extract from barn dust from farms (WO2006029685A1).

The present invention involves the use of a naturally derived complex preparation resulting from an effect-directed analysis (EDA) (FIG. 1), which has many advantages over the previously mentioned approaches. While still a complex mixture, the approach used for the present invention resulted in a highly deconvoluted fraction that is endotoxin free, sterile (by filtration and heat inactivation), and has defined molecular weight range. The resulting highly polar and endotoxin-free preparation is highly active, potent and has a robust biological response both in vivo and in vitro. Moreover, autoclaved Amish dust extracts demonstrated asthma protective properties both in vitro and in vivo, even though this procedure almost completely eliminates their proteolytic activity.

### BRIEF SUMMARY OF THE INVENTION

It is an objective of the present invention to provide compositions (or major components) of barn dust comprising bioactive fractions with sizes between 30 and 100 kDa (in particular, specific fractions including 28-64 kDa and 42-51.5 kDa), methods of use that allow for asthma and allergy prevention, and a novel in vitro cell assay to screen compounds for airway protectiveness as specified in the independent claims. Embodiments of the invention are given in the dependent claims. Embodiments of the present invention can be freely combined with each other if they are not mutually exclusive.

Children who grow up in a traditional farm environment rich in microbial exposures (e.g., dairy farms provide high exposure to animals and microbes) have a reduced risk of asthma and allergy and are protected from developing asthma and allergy. The significance of environmental exposures for asthma development is illustrated by epidemiologic studies demonstrating strong asthma protection in children from Alpine Europe raised on traditional farms. Children exposed to farming early in life have an asthma prevalence reduced over 5-fold compared to non-farming children (from 11.8% to 1.4%). Other studies have also shown that selected farm exposures contribute to the reduced asthma risk. For children in China, prevalence rates of wheeze and asthma were 1.0% and 1.1% in rural areas, being significantly lower than 7.2% and 6.3% found in an urban cohort, similar effects were also observed in adult populations. In urbanized environments, humans are exposed to vast numbers and types of inorganic and organic chemicals from endogenous and exogenous origins, with many chemicals being implicated in the increase of conditions such as asthma and allergies.

However, in North America, asthma prevalence in two farming communities (Amish and Hutterite) is strikingly different. In separate studies, 5.2% in Amish school children had asthma versus 11.5% in Hutterite school children. The main differences between these two groups, as far as living conditions, is that the Amish live on single family farms, utilize horses for transportation and fieldwork and practice traditional farming, while the Hutterites use industrial technology and live on communal farms. As such, not all farming environments provide the same protective effects against the development of asthma. The composition of dust in varied environments plays a major role in the onset of asthma and allergies or prevention thereof. The “farm effect” can be explained to a large extent by the child's early life contact with farm animals, in particular cattle.

Exposure to certain components of the farm environment has consistently been shown to explain the reduced risk of asthma and allergies: contact with livestock, mostly cattle, and contact with animal feed such as hay, straw and silage. Other differences in lifestyle, such as duration of breastfeeding, family size, pet ownership, other dietary habits, parental education or a family history of asthma and allergies did not account for the protective farm effect. These findings suggest that exposures encountered in animal sheds, in particular cattle barns, play a major role in asthma protection.

The present invention uses an effect-directed approach for chemical deconvolution of a highly active and potent extract capable of suppressing asthma and allergy in vivo by inducing immunological effects that closely recapitulate the immunological features relevant to asthma and allergy protection. The preparation and characterization of a complex mixture of agents derived from an aqueous farm dust extract deconvoluted to 0.4% of its initial total organic carbon (TOC) content, with a defined molecular weight range (e.g., 51.5-42 kDa) that is heat inactivated, sterile filtered, and endotoxin free is described herein. Preparations were evaluated using an in vivo model of ovalbumin (OVA)-induced airway hyperresponsiveness (AHR) and lung eosinophilia to assess asthma- and allergy-protective activity following intranasal administration. As few as 5 administrations of protective extract after OVA sensitization and before OVA challenge were sufficient to significantly suppress AHR, lung eosinophilia, OVA-specific IgE, and IL13 levels in BAL. These results highlight the therapeutic potential of preparations from farm dust extracts.

The present invention features a barn dust composition with asthma-protective properties. The barn dust composition may comprise bioactive fractions extracted from barn dust. In some embodiments, the bioactive fraction comprises one or more proteins and one or more fatty acids.

The present invention may also feature a method of preventing or treating allergies or asthma in a subject in need thereof. The method may comprise administering a therapeutically effective amount of a barn dust composition comprising bioactive fractions extracted from barn dust to a subject. In some embodiments, the bioactive fraction comprises one or more proteins and one or more fatty acids.

The present invention may further feature an in vitro method to screen compounds in barn dust extracts or barn dust fractions or barn dust sub-fractions thereof for airway protectiveness using 16HBE14o− epithelial cells. In some embodiments, the method comprises obtaining differentiated, confluent 16HBE14o− cells cultured in trans-wells plates (for apical/basal polarization). In other embodiments, the method comprises stressing a portion of the wells with the differentiated, confluent 16HBE14o− cells by culturing them in a serum-free medium. In further embodiments, the method comprises exposing the non-stressed and stressed cells to barn dust extracts, barn dust fractions or barn dust sub-fractions. In some embodiments, the method comprises measuring trans-epithelial electrical resistance (TEER) after a period of time. In further embodiments, the method comprises expressing results as % activity, with 100% activity corresponding to complete inhibition of the loss of TEER observed in epithelial cells cultured in serum-free medium alone. In some embodiments, the barn dust extracts, barn dust fractions or barn dust sub-fractions are protective of airways when 50% activity in the TEER assay is observed.

One of the unique and inventive technical features of the present invention is the specific fraction of farm barn dust extract, with the fraction size ranging between 30 and 100 kDa, particularly 28 to 64 kDa and 42 to 51.5 kDa fractions. Surprisingly, these fractions are still bioactive even in the presence of proteases and absence of endotoxins or arabinogalactan. These fractions also comprise at least eight (8) proteins, despite reported allergenic activity, that when combined, have asthma-protective properties including inhibition of AHR, airway eosinophilia, Th2 cytokine expression (including interleukins 4, 5, 10, and 13), and antigen-specific IgE. Without wishing to limit the invention to any theory or mechanism, it is believed that the specific fractions (in particular, specific 42-51.5 kDa fraction) comprising at least the eight (8) proteins advantageously provides for asthma and allergy protection. None of the presently known prior references or work has identified these particular eight (8) proteins and the specific 42-51.5 kDa fraction of barn dust extract to have airway protectiveness using in vivo mouse model and the novel in vivo and in vitro assays of the present invention described herein. Although some of the eight (8) proteins have been reported to have allergenic activity, in contrast to immunotherapy, a general protection appears to be conferred by these substances and not specific to these allergens. This mechanism is therefore profoundly different from that of conventional immunotherapy.

Furthermore, the prior references teach away from the present invention. For example, with the increased understanding about the importance of microbial diversity and its relationship with asthma and allergies, many studies have pointed to environmental and host microbiome interactions and health outcomes, with different species being associated with asthma and allergy protection. In other words, the prior art teaches the inclusion of endotoxin for asthma protection. Contrary to the prior art, the present invention here features an endotoxin-free, proteolytically active, narrowly defined, and standardized formulation. The present invention presents a clearer path for possible therapeutic use. The approach described herein, provides evidence that a naturally derived complex preparation is sufficient to provide protection in physiologically relevant in vitro and in vivo models. The result is surprising because the fraction protects from asthma despite having proteolytic activity, which is known to be associated with induction of asthma. Allergenic character of identified proteins is also surprising as it is considered more as an asthma risk than protection.

Previous approaches to isolate and identify protective agents of asthma and allergies from environmental sources rely on identification of single microbial species or molecular structures. Under this perspective, putative agents were previously identified as protective agents with heterogeneous results regarding protection and mechanism of action, notably microbial strains such as Acinetobacter lwoffii and Lactococcus lactis, Bacillus licheniformis spores, the polysaccharide Arabinogalactan, N-glycolylneuraminic acid (Neu5Gc), and Penicillium chrysogenum. Another common approach is the reliance on raw environmental extracts or on general parameters like proteolytic activity or broad classes of structures like endotoxins, or subclass Bacteroides endotoxins.

The present invention features a composition comprising a bioactive barn dust extract with asthma-protective properties. In preferred embodiments, the composition comprises bioactive fractions having a molecular weight between about 30 kDa and 100 kDa, preferably about 28 kDa to 64 kDa or about 42 kDa to 51.5 kDa. The bioactive fractions comprise bioactive components or molecules comprising at least one protein selected from a group of eight proteins comprising OBP, allergen Bos d2, interferon gamma, provicilin, vicilin (14 kDa component), beta-conglycinin, sarcoplasmic calcium-binding protein, and/or MATH domain At3g58400.

The present invention further features a method of preventing or treating a disease, particularly asthma and/or allergy-related conditions. The method comprises administering (e.g., intranasal administration) a therapeutically effective amount of a composition comprising a bioactive barn dust extract to a subject. In preferred embodiments, the bioactive barn dust comprises bioactive fractions having a molecular weight between about 30 kDa and 100 kDa, preferably about 28 kDa to 64 kDa or 42 kDa to 51.5 kDa. The bioactive fractions comprise bioactive components or molecules comprising at least one protein selected from a group of eight proteins comprising OBP, allergen Bos d2, interferon gamma, provicilin, vicilin (14 kDa component), beta-conglycinin, sarcoplasmic calcium-binding protein, and/or MATH domain At3g58400.

The present invention also features a novel in vitro method to screen compounds for airway protectiveness using 16HBE14o− epithelial cells to form polarized cell layers to study airway barrier function, solute transport, and responses to stress. The method comprises: providing differentiated, confluent 16HBE14o− cells cultured in trans-wells plates (for apical/basal polarization); stressing a portion of the differentiated, confluent 16HBE14o− cells by culturing them in a serum-free medium; exposing the non-stressed and stressed cells to a compound; measuring trans-epithelial electrical resistance (TEER) after 3, 12, 24, 48, or 72 hours; and expressing results as % activity, with 100% activity corresponding to complete inhibition of the loss of TEER observed in epithelial cells cultured in serum-free medium alone.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the experimental pipeline used to develop the present invention.

FIG. 2A shows a traditional, in vivo 39-day murine model Ovalbumin (OVA)-induced asthma protocol. Amish dust extracts (5 mg dust equivalent/treatment) were instilled evenly into the two nostrils (intranasal (i.n.)) every 2-3 days (14 times total) from day 0 to day 32 into 7-8 week old Balb/c mice (Envigo) that were sensitized intraperitoneally (i.p.) with ovalbumin (OVA: grade V, Sigma, 20 μg)-Alum (Pierce) at day 0 and 14, and challenged i.n. with OVA (50 μg) at day 28 and 38. A group of mice received saline at the time of treatment, sensitization and challenge. Terminal assessments (airway resistance, by the forced oscillation technique, BAL eosinophilia and cytokines) were performed at day 39.

FIG. 2B shows an in vivo 17-day murine model to test the activity of Amish extracts and their fractions. This model, an abbreviated version of the one described in FIG. 2A, leads to robust AHR, BAL eosinophilia and Th2 cytokine production in the lung, all measured at day 17. Amish dust extracts/fractions are administered 8 times over a 14-day period.

FIGS. 3A, 3B, and 3C show the therapeutic potential of Amish dust extracts. FIG. 3A shows a short OVA protocol to assess the ability of unfractionated Amish dust extracts to suppress established airway inflammation and asthma. OVA was used for i.p. sensitization (50 mg) and challenge (100 mg). Amish dust extracts were used at 5 mg dust equivalent/treatment. FIG. 3B shows airway resistance in response to increasing doses of nebulized methacholine as measured on a FlexiVent FX at day 20. FIG. 3C shows the percent cells in BAL of mice treated with OVA with or without Amish dust extract. Statistical differences between OVA and OVA/Amish treatments were assessed by Student's t test.

FIGS. 4A and 4B show activity of Amish farm dust fraction DB in the 16HBE14o− epithelial cell assay. FIG. 4A shows extracts were fractionated by size-exclusion chromatography (SEC) using the elution times indicated on the x axis. Fractions were named as indicated at the top of the graph. The labeled black and red lines show spectrophotometric readings obtained at 260 and 280 nm, respectively. FIG. 4B shows the activity of unfractionated extracts (Amish 4, IowaB) and fraction DB as protection of human 16HBE14o− airway epithelial cells from a serum starvation-induced decrease in trans-epithelial electrical resistance (TEER). Differentiated, confluent 16HBE14o− cells are stressed by culture in serum-free medium. At the same time, cells are exposed to dust extracts (1 mg/well). TEER is measured 24 hr later using a Millicell-ERS Volt-Ohm-meter (Millipore) and is expressed as Ωcm². GM-: cultures incubated in serum-free medium alone.

FIGS. 5A and 5B show inhibition of OVA-induced AHR by an endotoxin-depleted unfractionated Amish aqueous extract (DX). Activity of these preparations after intranasal administration was tested in the in vivo model presented in FIG. 2B. FIG. 5A shows measurements of airway resistance in response to methacholine challenge. FIG. 5B shows measurements of total BAL cellularity are shown in the left and right panel, respectively. Statistical differences were assessed by Student's t test. In the left panel: *p≤0.01, **p≤0.005, ***p≤0.00001.

FIGS. 6A, 6B, and 6C show separation analyses of an Amish dust sample. FIG. 6A shows SEC separation of an Amish dust sample. FIG. 6B shows subsequent analysis using liquid chromatography-quadrupole time of flight (LC-QTOF) for an C18 column. FIG. 6C shows subsequent analysis using LC-QTOF for a hydrophilic interaction liquid chromatography (HILIC) column.

FIGS. 7A and 7B show the effects of SEC fraction DB, KJ (WGA flowthrough) and KO (WGA retentate) on OVA-induced AHR and BAL eosinophilia. Activity of these preparations after intranasal administration was tested in the in vivo model presented in FIG. 2B. FIG. 7A shows a measurement of airway resistance in response to methacholine challenge (FlexiVent FX). FIG. 7B shows a measurement of % BAL cellularity. Statistical differences were assessed by Student's t test.

FIG. 8 shows extract deconvolution and generation of SEC fraction DB. The figure depicts the steps (dialysis of unfractionated extract, filtration, SEC) that led to generating fraction DB. Shown is also the level of mass concentration achieved in this process (to 0.51% of the original total carbon content).

FIGS. 9A and 9B show glycan-containing molecules mass spectrometry analysis. FIG. 9A shows principal components analysis (PCA) of raw sample, SEC fractions and lecithin-treated samples. FIG. 9B shows hierarchical clustering analysis (HCA) of entities and sample treatments for relevant glycan-containing samples.

FIGS. 10A and 10B show fraction DC and endotoxin-depleted DC, but not the endotoxin column eluate, inhibit both AHR and BAL eosinophilia. Activity of these preparations after intranasal administration was tested in the in vivo model presented in FIG. 2B. Measurements of airway resistance in response to methacholine challenge and total BAL cellularity are shown in the left and right panel, respectively. Statistical differences were assessed by Student's t test.

FIG. 11 shows Inhibitory activity of fractions DA, DD and DE on OVA-induced BAL eosinophilia. The activity of these preparations after intranasal administration was tested in the in vivo model presented in FIG. 2B. BAL cellularity is shown. Statistical differences were assessed by Student's t test. *p=0.0003; *p=0.001; ***p=0.01.

FIG. 12 shows analysis of Bos d2 in recombinant form or isolated from Amish dust extract fractions DA-DE. Loading: rBos d2: 62.5 ng/lane, fractions: 30 mg of dust equivalent/lane. Calculations: 5 mg of dust equivalents of DC contains ˜10.4 mg of Bos d2; 5 mg of dust equivalents of DB ˜2-5 mg of Bos d2.

FIG. 13 shows immunoprecipitation of Bd2 and OBP. ˜120 ng of OBP in 50 ug of DEQ of B extract->12-15 ug/5 mg of dust equivalents (DB and DC contain similar amounts).

FIGS. 14A and 14B show sugar residues and amino acid positioning for identified modifications on OBP (FIG. 14A) and Bos d2 (FIG. 14B) immunoprecipitated samples.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting

The term “barn dust” as used herein, relates to dust that can be, is, or has been collected from a barn. In some embodiments, the barn dust is collected from the floor or ground of a barn or from the top of a beam located within a barn. Without wishing to be bound by theory it is believed that barn dust comprises immunostimulatory substances derived from microorganisms, animals, plants, fungi, viruses and/or protozoa that are protective against allergies, asthma and/or other diseases disclosed herein. In preferred embodiments, the barn dust is from a farm. The origin of the barn dust is not limited to certain types of barns and can be any type of barn, including barns for any type of livestock such as cows, pigs, chicken, sheep, or horse. In preferred embodiments, the barn dust may be obtained from cow barns.

Also, the geographic location of the barn is believed to be not essential for the invention. Exemplary non-limiting geographic locations for a barn are the continental and non-continental United States of America, Europe, including the member states of the European Union, such as Germany, France, Austria, Switzerland, Czech Republic, Poland, the Netherlands, Belgium, Luxemburg, Spain, Portugal, Italy, etc. Barn dust can be collected by any suitable method known to the person skilled in the art, optionally by applying any type of collection system that is suitable for collecting barn dust. Barn dust can for example be collected by sweeping, vacuuming, or swiping. Barn dust can also be collected by filtration of barn air, for example by using the membrane filter or a granular material that is capable of adsorbing barn dust. Barn dust can also be collected using an impinge or an impactor, such as a cascade impactor.

In some embodiments, the barn dust is autoclaved and filtered through a 0.22-micron filter for sterility.

The term “barn dust extract” as used herein preferably refers to a composition that is obtainable by the methods disclosed herein and may refer to both, a solution or suspension, or a dry composition.

The term “fractions” as used herein refers to fractions that can be obtained by fractionating a mixture according to the elution time, molecular weight and/or size of the molecules comprising the mixture.

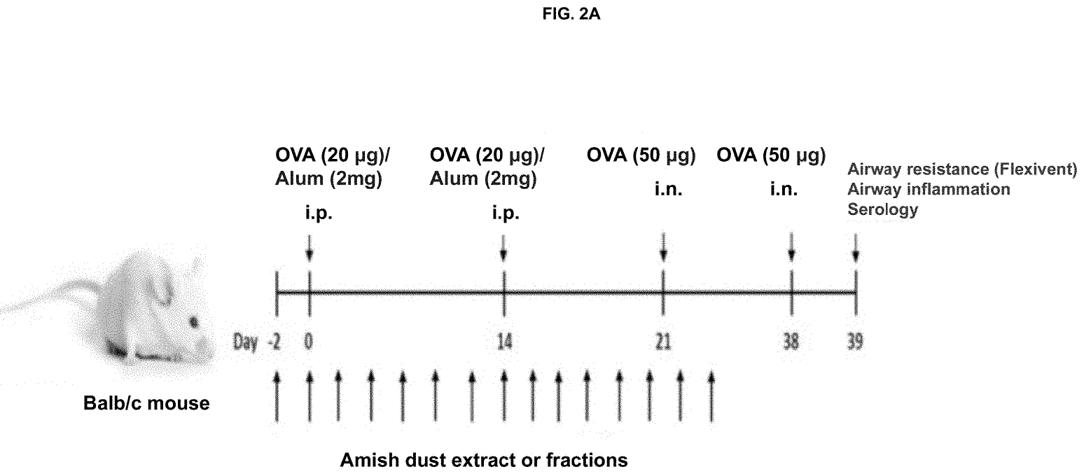

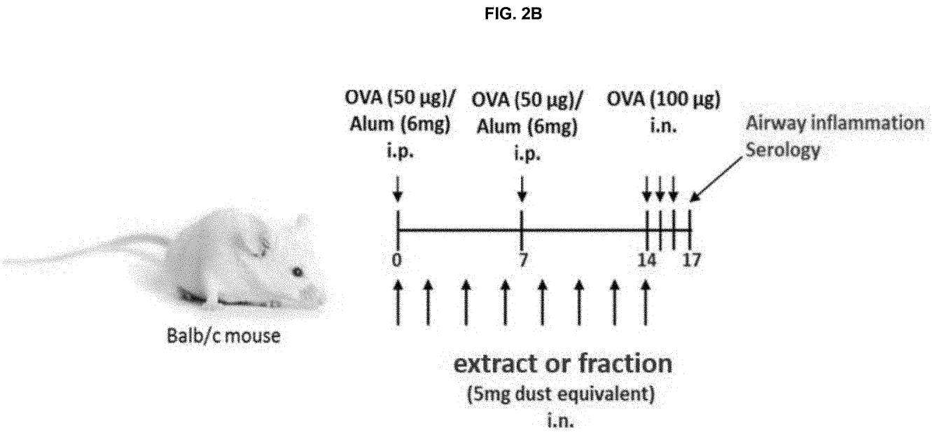

The term “bioactive fraction” as used herein refers to a fraction active in vitro in the TEER epithelial cell assay and in vivo in the mouse model shown in FIG. 2B.

As used herein ‘bioactive” refers to compositions that inhibit airway hyperresponsiveness (AHR) and/or broncho-alveolar lavage (BAL) eosinophilia and trans-epithelial electrical resistance (TEER) loss.

As used herein, “administering” and the like refer to the act physically delivering a composition or other therapy (e.g. a bioactive fraction or molecule of bioactive barn dust extract) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof. In preferred embodiments, nasal administration of the fractions and/or extracts is critical for bioactivity of the fractions and/or extracts.

A composition can also be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering a composition as described herein, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human under medical care or animals under veterinary care. Non-limiting examples of a subject comprise a baby, an infant, or a pregnant woman.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “effective amount” as used herein refers to the amount of a therapy or medication (e.g., bioactive composition, fraction, or molecule of bioactive barn dust extract provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., asthma), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of bioactive composition, fraction, or molecule of bioactive barn dust extract described herein is an amount sufficient enough to provide a therapeutic benefit in the treatment or management of asthma or an allergy-related condition, or to delay or minimize one or more symptoms associated with the presence of asthma or an allergy-related condition. A therapeutically effective amount of an agent (e.g., OBP) described herein, means an amount of therapeutic agent, alone or in combination with other therapies, which provides a therapeutic benefit in the treatment or management of asthma or an allergy-related condition. The term “therapeutically effective amount” can encompass an amount that improves overall therapy, reduces or avoids symptoms or causes of asthma or an allergy-related condition, or enhances the therapeutic efficacy of another therapeutic agent.

In some embodiments, a therapeutically effective amount may be calculated based on the method of administering the barn dust composition. In other embodiments, the calculation for a therapeutically effective amount depends on how much barn dust a subject naturally encounters.

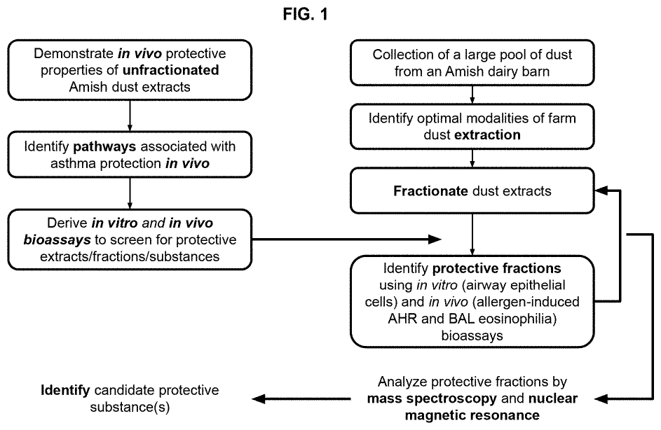

Referring now to FIGS. 1-14B, the present invention features compositions and methods for the prevention and treatment of asthma and allergies. The present invention may further feature an in vitro method for screening compounds in barn dust extracts or barn dust fractions or barn dust sub-fractions thereof, for airway protectiveness.

The present invention features a dust composition with asthma-protective properties, the barn dust composition comprising one or more bioactive fractions extracted from barn dust, said one or more bioactive fractions comprising one or more proteins and one or more fatty acids.

In some embodiments, the one or more proteins comprising the bioactive fraction are microbial proteins. In some embodiments, the one or more proteins comprising the bioactive fraction are plant proteins. In some embodiments, the one or more proteins comprising the bioactive fraction are animal proteins.

In some embodiments, the one or more proteins comprise transport proteins (e.g., one or more transport proteins). In some embodiments, the one or more proteins bind ligands. In some embodiments, the one or more proteins bind one or more fatty acids. In some embodiments, the one or more proteins carry one or more fatty acids. In some embodiments, the one or more transport proteins bind one or more fatty acids. In some embodiments, the one or more transport proteins carry one or more fatty acids.

In some embodiments, the one or more proteins bind one or more fatty acids. In some embodiments, the one or more transport proteins bind one or more fatty acids. In some embodiments, the one or more proteins bind one or more proteins. In some embodiments, the one or more transport proteins bind one or more proteins. In some embodiments, the one or more proteins bind one or more protein complexes. In some embodiments, the one or more transport proteins bind one or more protein complexes. In some embodiments, the one or more proteins form dimers which creates a pocket for a ligand to bind. In some embodiments, the one or more transport proteins form dimers which creates a pocket for a ligand to bind.

In some embodiments, the one or more transport proteins are selected from a group consisting of Bos 2d, odorant-binding protein, interferon gamma, provicilin, vicilin (14 kDa component), beta-conglycinin, sarcoplasmic calcium-binding protein, and MATH domain At3g58400.

In some embodiments, the one or more fatty acids comprise one or more polyunsaturated fatty acids (PUFAs). Non-limiting examples of PUFAs may include but are not limited to 11-HpOME, 9,12-dihydroxy stearic acid, 12-oxo-10E-octadecenoic acid, 9-hydroperoxy-10E,12,15Z-octadecatrienoic acid, 9,10,13-trihydroxy-11-octadecenoic acid, 9-hydroxy-10-oxo-12-octadecenoic acid, 9R,10S,18-trihydroxy-stearic acid, α-12(13)-EpODE, 3-keto palmitic acid, 6E,8E,14E-Hexadecatriene-10,12-diynoic acid, 9,12-Octadecadiynoic acid, 8-Octadecenoic acid, 10-hydroxy-12-oxo,13-hydroxy-9Z-octadecenoic acid, 6-ethyl-tetradecanoic acid, 11,15-dimethyl-hexadecanoic acid, or 11-hydroperoxy-12,13-epoxy-9-octadecenoic acid.

In some embodiments, the PUFAs are selected from a group consisting of 11-HpOME, 9,12-dihydroxy stearic acid, 12-oxo-10E-octadecenoic acid, 9-hydroperoxy-10E,12,15Z-octadecatrienoic acid, 9,10,13-trihydroxy-11-octadecenoic acid, 9-hydroxy-10-oxo-12-octadecenoic acid, 9R,10S,18-trihydroxy-stearic acid, α-12(13)-EpODE, 3-keto palmitic acid, 6E,8E,14E-Hexadecatriene-10,12-diynoic acid, 9,12-Octadecadiynoic acid, 8-Octadecenoic acid, 10-hydroxy-12-oxo,13-hydroxy-9Z-octadecenoic acid, 6-ethyl-tetradecanoic acid, 11,15-dimethyl-hexadecanoic acid, 11-hydroperoxy-12,13-epoxy-9-octadecenoic acid.

In preferred embodiments, the bioactive fractions of the barn dust are extracted using a polar solvent comprising water, and/or an aqueous solution. A non-limiting example of the aqueous solution comprises a solution of sodium chloride, preferably normal saline, distilled water, PBS/Saline, or 80% water 20% methanol in 20 mM ammonium bicarbonate.

In some embodiments, the bioactive fractions of the barn dust are extracted at room temperature (about 20° C.). In other embodiments, the bioactive fractions of the barn dust are extracted at about 30° C. In other embodiments, the bioactive fractions of the barn dust are extracted at about 50° C. In other embodiments, the bioactive fractions of the barn dust are extracted at about 80° C. In other embodiments, the bioactive fractions of the barn dust are extracted at about 121° C.

In some embodiments, the bioactive fractions of the barn dust are extracted at about standard pressure (1 atm). In some embodiments, the bioactive fractions of the barn dust are extracted at about 2 atm. In some embodiments, the bioactive fractions of the barn dust are extracted at about 50 atm. In some embodiments, the bioactive fractions of the barn dust are extracted at about 100 atm.

In other preferred embodiments, the asthma-protective properties comprise preventing, suppressing, and/or abrogating airway hyperresponsiveness (AHR) and/or broncho-alveolar lavage (BAL) eosinophilia, and/or reducing serum IL-13, IL-5, IL-4 and/or IgE levels specifically in the lung and airway mucosa, particularly due to allergens. Non-limiting examples of allergens comprise allergenic proteins, ovalbumin, house dust mites, cockroaches, Alterneria, pollen, pet dander, and/or other environmental-, animal-, and/or plant-based allergens.

In some embodiments, the bioactive fractions comprise a molecular weight of about 28 to 64 kDa, about 28 to 42 kDa, about 42 to 51.5 kDa, about 50 to 70 kDa. In some embodiments, the bioactive fraction comprises an unfractionated barn dust extract. In some embodiments, the barn dust may comprise an Amish farm barn dust, and Hutterite farm barn dust. Non-limiting examples of the barn comprise a cow barn or shed, or a barn or shed from any traditional dairy farm comprising at least one cow.

In some embodiments, the barn dust does not contain living organisms. In other embodiments, the barn dust extract is a solubilized material. In some embodiments, the barn dust extract has less than 2.0% of organic content of the original sample. In some embodiments, the barn dust composition comprises less than 2.0% total organic carbon content, less than 0.2% endotoxins, less than 0.2% lipopolysaccharides, or a combination thereof as compared to an original barn dust sample.

In preferred embodiments, the barn dust extract is thermostable with no loss of activity with temperatures up to at least 121° C. In other embodiments, the barn dust extract is stable with no loss of activity with pressure to at about 102 atm to 108 atm, preferably, to at least 103 atm. In some embodiments, the barn dust extract is endotoxin-free, comprising less than 0.2% endotoxin. In other embodiments, the barn dust extract is essentially endotoxin-free (e.g., the barn dust extract comprises less than 0.2% endotoxin). In some embodiments, the barn dust extract is active in the presence of proteases. In some embodiments, the barn dust extract is a solubilized material and/or has less than about 2.0% of organic carbon content of the original sample. Non-limiting examples of total organic carbon (TOC) comprise 2.17% for the 64-28 range, 0.52% for the 42-51.5 range, and 0.4% for aforementioned endotoxin free preparation.

In some embodiments, the barn dust extract has proteolytic activity. In other embodiments, the barn dust extract has no proteolytic activity.

As used herein, “endotoxin-free” refers to a negative score in a classical Limulus Amebocyte Lysate (LAL) assay. In some embodiments, the test sensitivity/limit of detection was 0.01 EU/mL.

In other preferred embodiments, the barn dust composition comprises glycosylated molecules. In some embodiments, the barn dust composition comprises less than 0.2% lipopolysaccharides. In further embodiments, the barn dust composition comprises glycoconjugates comprising glycoproteins, glycopeptides, peptidoglycans, glycolipids, glycosides, and/or lipopolysaccharides. In other circumstances, the barn dust composition comprises glycan-like molecules that are bioactive. In some embodiments, the barn dust composition comprises glycoproteins carrying small molecules including but not limited to polyunsaturated fatty acids (PUFAs). In some embodiments, the barn dust composition is not active in the presence of β-galactosidase.

In some embodiments, the barn dust composition is a pharmaceutical composition. In other embodiments, the pharmaceutical composition is in the form of a solution, an aerosol, a suspension, a lyophilisate, a powder, a tablet, a dragee, or a suppository. The composition can be used for nasal, inhalation, oral, conjunctival, subcutaneous, intraarticular, intraperitoneal, rectal, or vaginal administration. In preferred embodiments, nasal administration of the fractions and/or extracts is critical for bioactivity of the fractions and/or extracts. In other embodiments, the composition is a food additive, a food ingredient, or a composition suitable to be distributed in indoor air.

In preferred embodiments, the barn dust composition is manufactured for use in the prevention or treatment of a disease. In some embodiments, the barn dust composition is used for the prevention and treatment of a disease. Non-limiting examples of diseases comprise an allergic disease, asthma, a chronic inflammatory disease, and/or an autoimmune disease. The disease can be selected from the group consisting of hay fever, food allergy, asthma, urticaria, neurodermitis, atopy, including atopic sensitization and atopic dermatitis, contact eczema, psoriasis, diabetes type 1 or 2, multiple sclerosis, rheumatoid arthritis, diseases of the thyroid gland, including Hashimoto thyroiditis and Graves disease. In preferred embodiments, the disease is selected from the group consisting of atopy, including atopic sensitisation and atopic dermatitis, asthma and hay fever. The bioactive barn dust extract can be administered to a subject comprising a human or an animal. Non-limiting examples of a subject comprise a baby, an infant, or a pregnant woman.

In some embodiments, the composition is administered to infants or pregnant mothers as a preventative measure for the diseases and conditions described herein. In other embodiments, the composition is administered to children or adults as a treatment for the diseases and conditions described herein.

In preferred embodiments, the barn dust composition comprises one or more bioactive fractions, components, and/or molecules. In some embodiments, the barn dust composition prevents the development and/or treats the occurrence of asthma or allergy. In some embodiments, the barn dust composition reduces occurrence of asthma attacks or allergic reactions. In other embodiments, the barn dust composition treats the reaction to asthma triggers comprising physical exertion, allergies, and/or allergens. In some embodiments, the barn dust composition is used as a vaccine to prevent development of asthma or allergy. In other embodiments, the barn dust composition is used to treat allergies comprising allergic asthma, allergic rhinitis, allergic dermatitis, food allergies. The bioactive components or molecules can also be used to treat allergies comprising food allergies.

The present invention may also feature a method of preventing or treating allergies or asthma in a subject in need thereof. The method may comprise administering a therapeutically effective amount of a barn dust composition comprising one or more bioactive fractions extracted from barn dust to a subject. In some embodiments, the one or more bioactive fraction comprises one or more proteins and one or more fatty acids.

In preferred embodiments, the present invention features a method for preventing or treating a disease comprising administering a therapeutically effective amount of a bioactive barn dust extract to a subject. The bioactive barn dust extract comprises bioactive fractions having a molecular weight of about 30 kDa to about 100 kDa, preferably about 28 kDa to about 64 kDa or about 42 kDa to about 51.5 kDa. These bioactive fractions comprise bioactive components or molecules comprising at least one protein selected from a group of eight proteins comprising OBP, allergen Bos d2, interferon gamma, provicilin, vicilin (14 kDa component), beta-conglycinin, sarcoplasmic calcium-binding protein, and MATH domain At3g58400.

Non-limiting examples of allergies the method described herein may prevent or treat include but are not limited to soy allergies, peanut allergies, pollens, or a combination thereof. In other embodiments, allergies may include but are not limited to allergic asthma, allergic rhinitis, allergic dermatitis, food allergies, or a combination thereof.

In some embodiments, the method is for use as immunotherapy interventions for allergies and/or an underlying process of allergy that triggers asthma. In other embodiments, the methods described herein treat the underlying process of allergies that trigger asthma. In some embodiments, the method is used to modulate the immune system to prevent asthma or decrease the occurrence of asthma attacks. In some embodiments, the method is used to modulate the immune response to prevent asthma or decrease the occurrence of asthma attacks. In some embodiments, the method reduces the occurrence of asthma attacks and/or allergic reactions. In other embodiments, the method reduces the development of asthma or allergy. In preferred embodiments, the method treats the reaction to asthma triggers comprising physical exertion, allergies, and/or allergen.

In some embodiments, the method is used as a vaccine to prevent development of asthma or allergy. In other embodiments, the method is used to treat allergies including food allergies.

The present invention also features a novel in vitro method to screen compounds for airway protective properties using 16HBE14o− epithelial cells that form polarized cell layers to study airway barrier function, solute transport, and responses to stress. In some embodiments, the method comprises obtaining differentiated, confluent 16HBE14o− cells cultured in trans-wells plates (for apical/basal polarization). In other embodiments, the method comprises stressing a portion of the wells with the differentiated, confluent 16HBE14o− cells by culturing them in a serum-free medium. In further embodiments, the method comprises exposing the non-stressed and stressed cells to barn dust extracts, barn dust fractions or barn dust sub-fractions. In some embodiments, the method comprises measuring trans-epithelial electrical resistance (TEER) after a period of time. In further embodiments, the method comprises expressing results as % activity, with 100% activity corresponding to complete inhibition of the loss of TEER observed in epithelial cells cultured in serum-free medium alone. In some embodiments, the barn dust extracts, barn dust fractions or barn dust sub-fractions are protective of airways when 50% activity in the TEER assay is observed.

In some embodiments, a period of time may refer to 3 hours, 12 hours, 24 hours, 48 hours or 72 hours.

In some embodiments, the method described herein further comprises validating the barn dust extracts, barn dust fractions or barn dust sub-fractions found to be protective in the aforementioned in vitro method, in vivo in asthma mouse models.

In some embodiments, the asthma mouse models are administered the barn dust extracts, barn dust fractions or barn dust sub-fractions. In other embodiments, the barn dust extracts, barn dust fractions or barn dust sub-fractions are administered to the asthma mouse models by inhalation.

In some embodiments, the barn dust extracts, barn dust fractions or barn dust sub-fractions are administered once. In some embodiments, the barn dust extracts, barn dust fractions or barn dust sub-fractions are administered twice. In some embodiments, the barn dust extracts, barn dust fractions or barn dust sub-fractions are administered three times. In some embodiments, the barn dust extracts, barn dust fractions or barn dust sub-fractions are administered four times. In some embodiments, the barn dust extracts, barn dust fractions or barn dust sub-fractions are administered five times. In some embodiments, the barn dust extracts, barn dust fractions or barn dust sub-fractions are administered six times.

In some embodiments, the barn dust extracts, barn dust fractions or barn dust sub-fractions are administered over a one-day period. In some embodiments, the barn dust extracts, barn dust fractions or barn dust sub-fractions are administered over a five-day period. In some embodiments, the barn dust extracts, barn dust fractions or barn dust sub-fractions are administered over a ten-day period. In some embodiments, the barn dust extracts, barn dust fractions or barn dust sub-fractions are administered over a 17 day period. In some embodiments, the barn dust extracts, barn dust fractions or barn dust sub-fractions are administered over a 20 day period. In some embodiments, the barn dust extracts, barn dust fractions or barn dust sub-fractions are administered over a 30 day period.

In some embodiments, the barn dust extracts, barn dust fractions or barn dust sub-fractions are administered at various concentrations. In some embodiments, the barn dust extracts, barn dust fractions or barn dust sub-fractions are administered at a concentration between about 0.02 mg/well of dust equivalent to 4 mg/well of dust equivalent. In other embodiments, the barn dust extracts, barn dust fractions or barn dust sub-fractions are administered at a concentration between about 0.03 mg/well of dust equivalent to 3 mg/well of dust equivalent. In some embodiments, the barn dust extracts, barn dust fractions or barn dust sub-fractions are administered at a concentration of about 0.03 mg/well of dust equivalent. In some embodiments, the barn dust extracts, barn dust fractions or barn dust sub-fractions are administered at a concentration of about 0.1 mg/well of dust equivalent. In some embodiments, the barn dust extracts, barn dust fractions or barn dust sub-fractions are administered at a concentration of about 0.33 mg/well of dust equivalent. In some embodiments, the barn dust extracts, barn dust fractions or barn dust sub-fractions are administered at a concentration of about 1 mg/well of dust equivalent. In some embodiments, the barn dust extracts, barn dust fractions or barn dust sub-fractions are administered at a concentration of about 3 mg/well of dust equivalent.

As used herein, a “dust equivalent” refers to aqueous extracts prepared from unfractionated farm dust as follows: 100 mg of farm dust per one mL of endotoxin-free distilled water/saline was agitated for one hour (2000 rpm), centrifuged at 600×g (20 min, 4° C.) and then the supernatant was decanted. Concentration of the prepared extract was nominally assigned as 100 mg dust equivalent/mL.

In some embodiments, 100 mg of farm dust was combined with one mL of an endotoxin-free aqueous solution. In some embodiments, 150 mg of farm dust was combined with one mL of an endotoxin-free aqueous solution. In some embodiments, 50 mg of farm dust was combined with one mL of an endotoxin-free aqueous solution. In some embodiments, 25 mg of farm dust was combined with one mL of an endotoxin-free aqueous solution.

In some embodiments, the farm dust combined with the endotoxin-free aqueous solution is agitated for one hour. In some embodiments, the farm dust combined with the endotoxin-free aqueous solution is agitated for 30 minutes. In some embodiments, the farm dust combined with the endotoxin-free aqueous solution is agitated for 90 minutes. In some embodiment, the farm dust combined with the endotoxin-free aqueous solution is agitated for two hours.

In some embodiment, the farm dust combined with the endotoxin-free aqueous solution is centrifuged at about 600×g. In some embodiment, the farm dust combined with the endotoxin-free aqueous solution is centrifuged at about 300×g. In some embodiment, the farm dust combined with the endotoxin-free aqueous solution is centrifuged at about 500×g. In some embodiment, the farm dust combined with the endotoxin-free aqueous solution is centrifuged at about 800×g.

In some embodiment, the farm dust combined with the endotoxin-free aqueous solution is centrifuged for about 20 minutes. In some embodiment, the farm dust combined with the endotoxin-free aqueous solution is centrifuged for about 10 minutes. In some embodiment, the farm dust combined with the endotoxin-free aqueous solution is centrifuged for about 30 minutes.

In some embodiments, the farm dust combined with the endotoxin-free aqueous solution is centrifuged at about 4° C. In some embodiments, the farm dust combined with the endotoxin-free aqueous solution is centrifuged at about 8° C. In some embodiments, the farm dust combined with the endotoxin-free aqueous solution is centrifuged at about 12° C.

In some embodiments, the barn dust extracts, barn dust fractions or barn dust sub-fractions are validated when a decrease airway hyperresponsiveness (AHR) and/or broncho-alveolar lavage (BAL) eosinophilia is observed. In some embodiments, the barn dust extracts, barn dust fractions or barn dust sub-fractions are protective when a decrease airway hyperresponsiveness (AHR) and/or broncho-alveolar lavage (BAL) eosinophilia is observed.

In some embodiments, the barn dust extracts, barn dust fractions or barn dust sub-fractions are validated when the barn dust extracts, barn dust fractions or barn dust sub-fractions are effective in both the in vitro and in vivo methods described herein. In some embodiments, the barn dust extracts, barn dust fractions or barn dust sub-fractions are validated when the barn dust extracts, barn dust fractions or barn dust sub-fractions are when 50% activity in the TEER assay is observed in vitro and a decrease airway hyperresponsiveness (AHR) and/or broncho-alveolar lavage (BAL) eosinophilia is observed in vivo.

In other embodiments, the barn dust extracts, barn dust fractions or barn dust sub-fractions are validated and found protective when a statistically significant decrease in airway hyperresponsiveness (AHR) and/or broncho-alveolar lavage (BAL) eosinophilia is observed. In some embodiments, the barn dust extracts, barn dust fractions or barn dust sub-fractions are validated and found protective when a decrease in airway hyperresponsiveness (AHR) and/or broncho-alveolar lavage (BAL) eosinophilia is observed. In further embodiments, the protective activity of the barn dust extracts, barn dust fractions or barn dust sub-fractions is validated when a statistically significant decrease in airway hyperresponsiveness (AHR) and/or broncho-alveolar lavage (BAL) eosinophilia is observed. In further embodiments, the protective activity of the barn dust extracts, barn dust fractions or barn dust sub-fractions is validated when a statistically significant decrease in airway hyperresponsiveness (AHR) and/or broncho-alveolar lavage (BAL) eosinophilia is observed in vivo. In other embodiments, the protective activity of the barn dust extracts, barn dust fractions or barn dust sub-fractions is validated when a decrease in airway hyperresponsiveness (AHR) and/or broncho-alveolar lavage (BAL) eosinophilia is observed.

In some embodiments, measuring TEER comprises standard analytical technology for extract analysis. In other embodiments, the wherein extract analysis technology comprises electrical resistance.

In preferred embodiments, measuring trans-epithelial electrical resistance (TEER) at 3, 12, 24, 48 or 72 hours after differentiated, confluent 16HBE14o− cells are stressed by culture in serum-free medium and exposed to different concentrations of dust extracts (1 mg/well). TEER is measured using a Millicell-ERS Volt-Ohm-meter (Millipore) and is expressed as Ωcm². In some embodiments, the method can be used for semi-high throughput screening, for example screening a large number of samples in a short period of time (e.g., 24-48 samples in 1, 2 or 3 days, or 1-3 plates each day). The method also may allow semi-quantitative comparisons of activity through dose-response curves. In some embodiments, the method further comprises use of gamma delta T lymphocytes (γδT or gdT) cell bioassay.

In other embodiments, he method comprises: 1) providing differentiated, confluent 16HBE14o− cells cultured in trans-wells plates (for apical/basal polarization); 2) stressing a portion of the differentiated, confluent 16HBE14o− cells by culturing them in a serum-free medium; 3) exposing the non-stressed and stressed cells to a compound; 4) measuring trans-epithelial electrical resistance (TEER) after 3, 12, 24, 48 or 72 hours; and expressing results as % activity, with 100% activity corresponding to complete inhibition of the loss of TEER observed in epithelial cells cultured in serum-free medium alone. In some embodiments, the compound comprises an allergen. Non-limiting examples of an allergen comprises allergenic proteins, farm dust, barn dust, ovalbumin, house dust mites, cockroaches, Alternaria, pollen, pet dander, smoke, and/or other environmental-, animal-, and/or plant-based allergens. The barn dust may comprise unfractionated dust extracts or fractions and/or sub-fractions thereof. The concentration of the extracts/fractions can comprise of 3, 1, 0.33, 0.1, 0.03 mg/well (dust equivalent) or concentrations sufficient to stimulate a response.

### EXAMPLES

The following are non-limiting examples of the present invention. It is to be understood that said example is not intended to limit the present invention in any way. Equivalents or substitutes are within the scope of the present invention.

### Example 1: Robust Protective Effects of Amish Dust Extracts in Mice

FIG. 1 shows the experimental pipeline and strategy of the present invention. Large pools of dust from an Amish dairy barn were collected in 2012, 2015, and 2016/2017. Optimal modalities/chemical characterization (e.g., solvent polarity/water extraction, thermostability, non-volatile, endotoxin absence) of farm dust extraction were then identified and the dust extracts were fractionated. Protective fractions were identified using in vitro (e.g., airway epithelial cells) and in vivo [allergen-induced airway hyperresponsiveness (AHR) and broncho-alveolar lavage (BAL) eosinophilia; e.g., murine models of ovalbumin (OVA) as shown in FIGS. 2A and 2B; house dust mite (HDM), Alternaria-induced AHR and BAL eosinophilia, or gdT bioassays] and further fractionation of the protective fractions was performed to identify protective subfractions. Protective fractions and subfractions were then analyzed by mass spectroscopy (MS) and nuclear magnetic resonance (NMR) and candidate protective substances were identified using in vitro and in vivo bioassays. Concurrently, in vivo protective properties were demonstrated for unfractionated Amish dust extracts, pathways were identified that are associated with asthma protection in vivo, and in vivo and in vitro bioassays were derived to screen for protective extracts/fractions/substances.

Using the experimental strategy described in FIG. 1 and as described below, in virtually all in vivo experiments, inhalation of unfractionated aqueous Amish dust extracts led to significant suppression and often abrogation of AHR and BAL eosinophilia regardless of the allergen [OVA, HDM, or Alternaria], model [17 days, 31 days, or classic 39 days], mouse strain (Balb/c or C57BL6 mice), mouse gender, batch of dust extract, and season in which the dust was collected. Moreover, the activity of extracts prepared from dust collected in 2012, 2015, and 2016/2017 was essentially identical, and the activity of the extracts themselves is extremely stable. The in vivo protective effects of Amish dust products and the models used to assess them are therefore quite robust.

### Example 2: Administration Route and Extract Properties Required for Asthma and Allergy Protection

(1) The in vivo results mentioned above were obtained by intranasal administration of Amish aqueous dust extracts. In contrast, administration of an equivalent dose of extract by gavage failed to significantly suppress OVA-induced AHR and lung eosinophilia. (2) Inhalation of autoclaved (121° C. for 15 minutes) Amish aqueous extracts significantly reduced AHR (p=0.01), BAL IL-5 and IL-13 levels (p=0.001) and lung eosinophilia (p=0.000001) in the classic in vivo OVA model, showing that live microbes are not required for the protective effects of Amish farm dust extracts. (3) In a prevention model (e.g., giving the extract before allergen sensitization), just three inhalations of unfractionated Amish aqueous extract before the initial OVA sensitization (days −5 to 0) were sufficient to significantly (p=0.01) suppress OVA-induced lung eosinophilia assessed 31 days later, pointing to a prolonged protective effect of the extracts.

### Example 3: Therapeutic Potential of Amish Dust Extracts

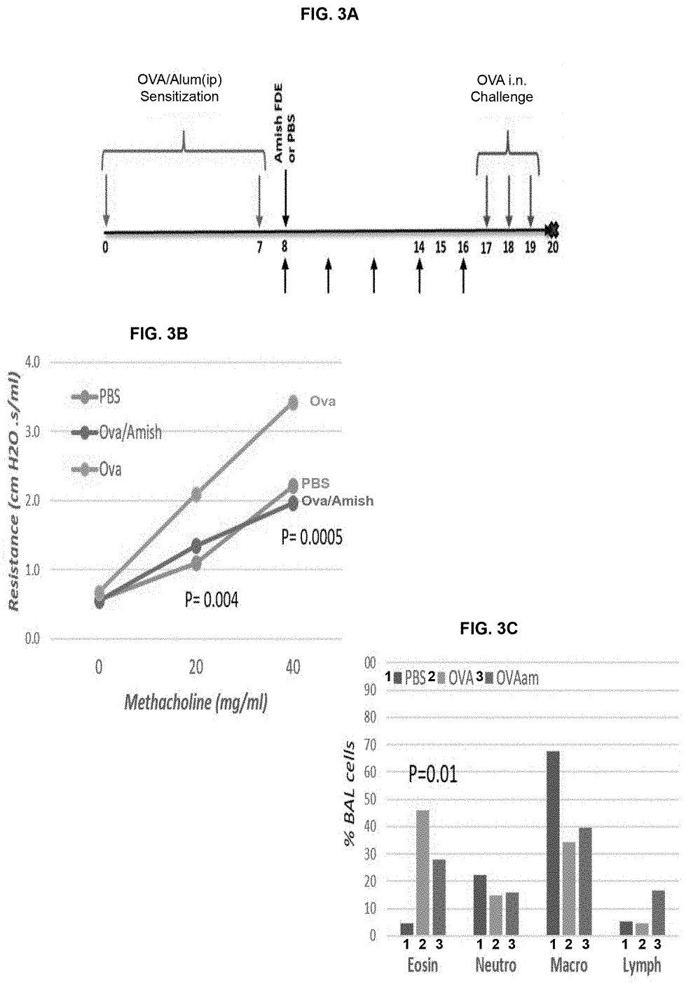

In a 17-day protocol, as few as five (5) administrations of Amish aqueous dust extract after OVA sensitization and before OVA challenge (FIG. 3A) were sufficient to significantly suppress AHR (FIG. 3B), lung eosinophilia (FIG. 3C), and OVA-specific IgE (p=0.02, not shown). Similar results were obtained in a 49 days protocol (not shown). IL13 levels in BAL were also significantly (p=0.01) decreased. These results highlight the therapeutic potential of Amish farm dust extracts.

### Example 4: A Novel In Vitro Epithelial Cell Bioassay for Airway Protection

In vitro bioassays are required that can screen a substantial number of samples in a short period of time and provide reliable information about their protective activity. The present invention discloses a new assay that monitors the ability of farm dust constituents to dose-dependently protect human 16HBE14o− airway epithelial cells from stress-induced loss of trans-epithelial electrical resistance (TEER).

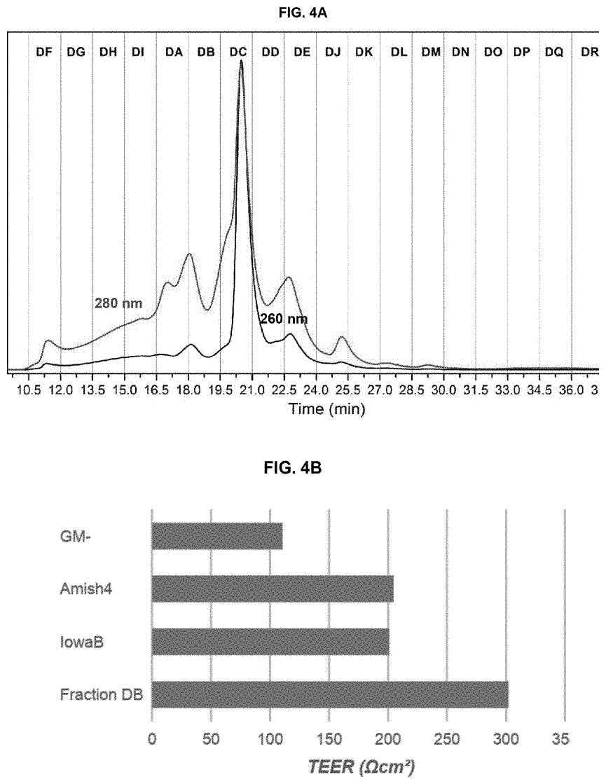

16HBE14o− cells2 form polarized cell layers in vitro and are excellent models to study airway barrier function, solute transport and responses to stress. In the assay of the present invention, differentiated, confluent 16HBE14o− cells cultured in trans-wells (for apical/basal polarization) are stressed by culturing them in serum-free medium. At the same time, cells are exposed to unfractionated Amish dust extracts or fractions and sub-fractions thereof at different concentrations (e.g., 3, 1, 0.33, 0.1, 0.03 mg/well of dust equivalent). Trans-epithelial electrical resistance (TEER) can be measured after 3, 12, 24, 48 or 72 hours; and results are expressed as % activity, with 100% activity corresponding to complete inhibition of the loss of TEER observed in epithelial cells cultured in serum-free medium alone. This assay is suitable for semi-high throughput screening (>20 extracts or fractions/week) and allows semi-quantitative comparisons of activity through dose-response curves. Samples active in this in vitro assay often also have protective activity in vivo, whereas products from non-protective environments fail (data not shown). Examples of the results from this assay are shown in FIGS. 4A and 4B demonstrating that the 16HBE14o− airway epithelial cell assay provides a robust, time-effective in vitro tool to screen Amish dust-derived materials for airway protective properties.

### Example 5: Highlights of Chemical Characterization of Amish Dust Extracts, Fractions and Substances

Extraction solvent selection and extraction efficiency: The resulting components from extracts of environmental samples are highly dependent on the solvent used for extraction. Recovery of bioactive substances from Amish dust samples was tested in a range of solvents with different characteristics (given here by polarity index (P′): water (P′ 10.2), methanol (P′ 5.1), chloroform (P′ 4.1), methylene chloride (P′ 3.1) and hexane (P′ 0.1). Of all resulting samples, only those extracted with water showed biological activity, demonstrating highly polar characteristics of bioactive substances. Second and third sequential aqueous extraction of the same sample did not show significant biological activity reinforcing the polar characteristic of active extracts and a high efficiency of selected method for extraction and further deconvolution.

Extract thermostability and volatility Extract response to different temperatures and possible volatility of substances of interest were tested by treating samples at different temperatures (25° C., 80° C. and 121° C.). Bioactivity was maintained in all temperature conditions. Effects of different extraction pressures were also tested (1 atm, 2 atm and 103 atm) with no loss of activity. Total dryness of samples with loss of volatile compounds also did not affect the biological response of the extract.

Living microorganisms and particle presence: The necessity for living organisms and/or particles to mediate biological activity was tested by sample filtration (unfiltered, 1.2 μm, 0.7 μm, 0.2 μm), autoclaving the extracts (2 atm, 121° C.), and submitting samples to high pressure and temperature (103 atm and 80° C.). All tests showed that living organisms are not necessary to maintain activity of the extracts. Filtration of samples also demonstrates that a solubilized material is responsible for bioactivity.

Molecular size distribution: Fractionation by molecular size is an initial step in the purification and isolation of bioactive components. Diafiltration procedures using different molecular weight cut-off membranes (3.5 kDa, 10 kDa, 30 kDa and 100 kDa) were used to identify an initial molecular size range of activity in aqueous farm dust extracts. With this procedure the selection of an ideal preparative size exclusion chromatography (SEC) column was possible (FIG. 4A), as the response observed for in vivo models and by treating epithelial cells in vitro (FIG. 4B) was mostly limited to the 30-100 kDa range. With the introduction of preparative SEC, a finer molecular weight range for bioactive components (28-64 kDa) was identified. This procedure efficiently enriches for bioactive substances of interest, eliminating 98.3% of the organic carbon content of the original sample. Use of a preparative separation was essential for a standardization of bioactive extract preparation and scale up of procedures.

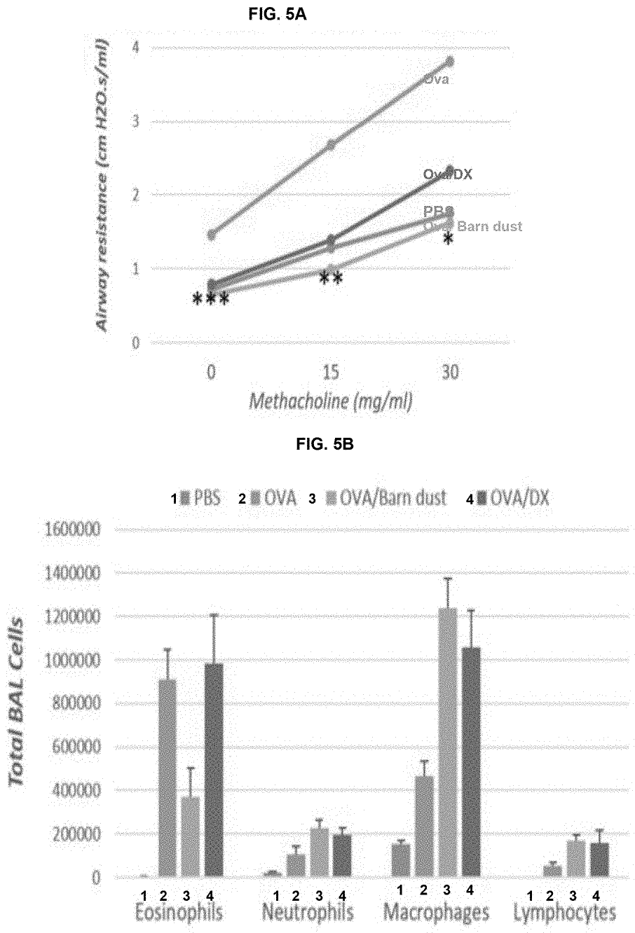

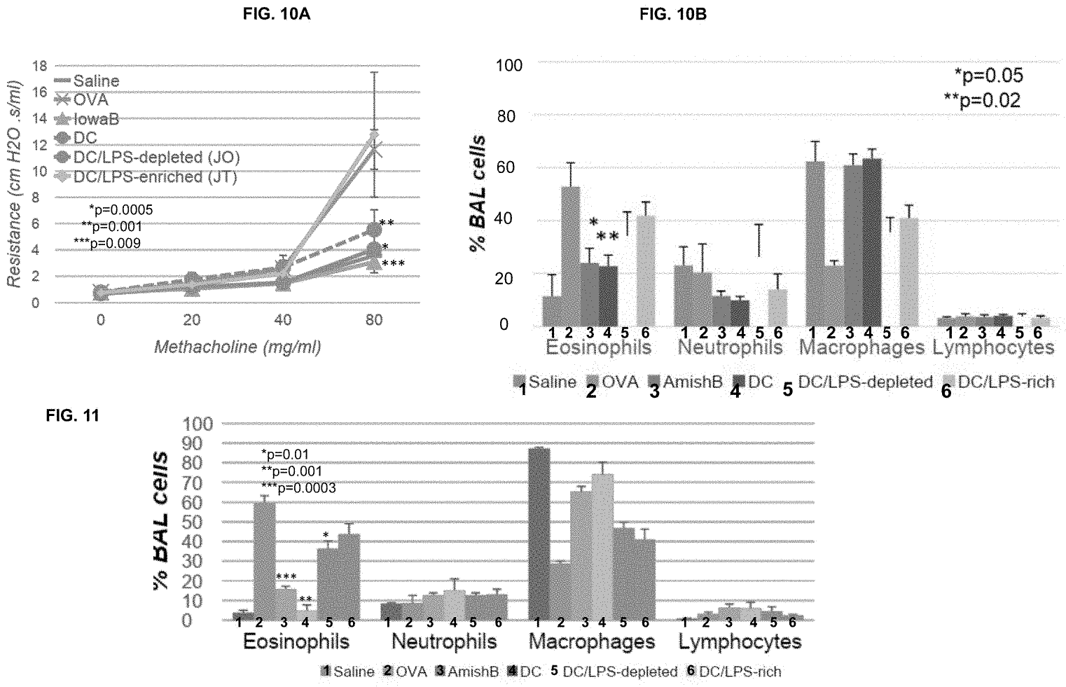

Endotoxin removal: To assess a potential role of endotoxin, which is abundant in the Amish environment and is known to be present in protective aqueous extracts, unfractionated extracts were thoroughly depleted of endotoxin by adsorption on a poly-(ε-lysine) resin. A limulus assay followed by mass spectrometry showed that >99.9% of endotoxin was removed by this approach. Endotoxin-depleted extracts were still active in vitro (FIG. 4B) and blocked AHR but not BAL eosinophilia in vivo (FIGS. 2B, 5A, 5B). Similar results were obtained with endotoxin-depleted SEC fraction DC (FIGS. 10A, 10B) while endotoxin-depleted SEC fraction DB was able to inhibit BAL eosinophilia (not shown).

Enzymatic digestion: To study some of the molecular characteristics of the extracts, different enzymes (Table 1) were used, based on molecular features identified during sample fingerprinting performed using liquid chromatography-quadrupole time of flight (LC-QTOF) analysis.

TABLE 1
Enzymes, and Corresponding Reaction Catalyzed,
used for Digestion of Amish Dust Samples.

| | | | Reaction |
|---|---|---|---|
| EC # | Enzyme | Organism | catalyzed |
| EC | Endo-1,4-β- | A. niger | Hydrolysis of (1->4)- |
| 3.2.1.89 | galactanase | | β -D-galactosidic |
| | | | linkages in type I |
| | | | arabinogalactans |
| EC | β-Galactosidase | K. lactis | Hydrolysis of terminal |
| 3.2.1.23 | | | non-reducing β -D- |
| | | | galactose |
| | | | residues in |
| | | | β-D-galactosides |
| EC | Proteinase K, | T. album | Broad-spectrum |
| 3.4.21.64 | immobilized | | serine protease |

After enzymatic digestion, Amish dust samples were processed by SEC to remove the added enzymes, and the resulting extracts were assessed for biological activity in the airway epithelial cell assay. Addition of a broad-spectrum serine protease (Proteinase K) at concentrations up to 500 U was unable to reduce the biological activity of the extract.

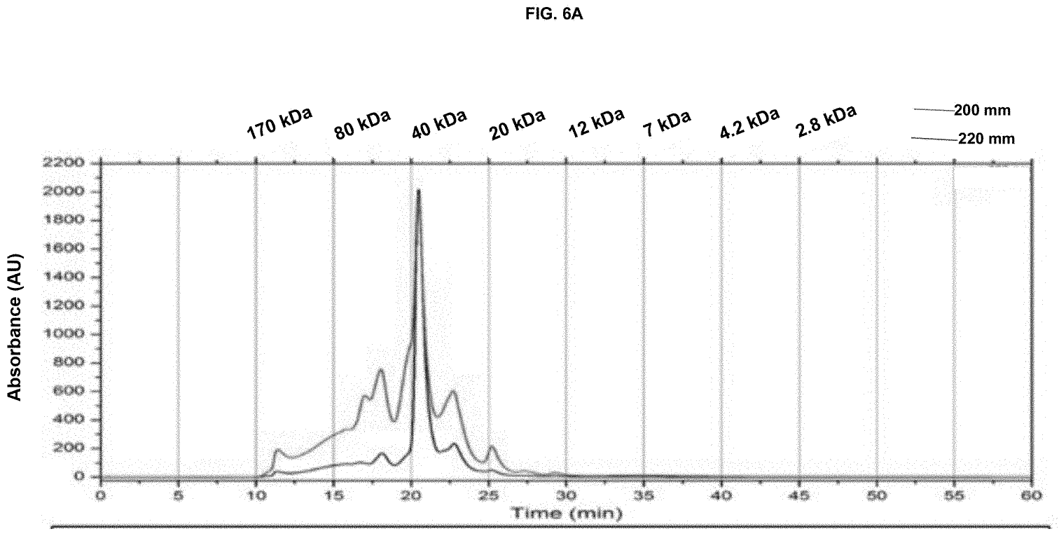

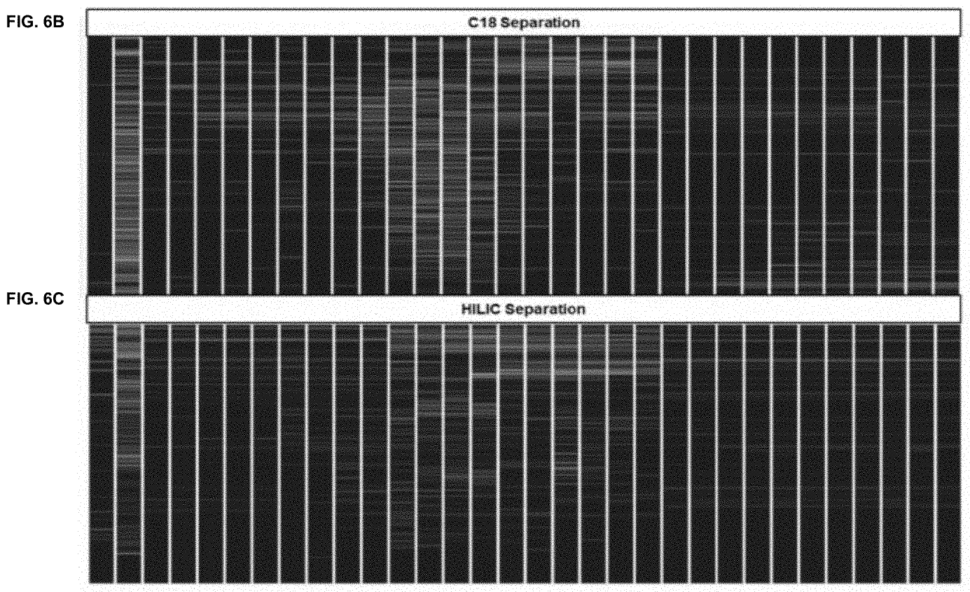

LC-QTOF structural identification of candidate substances: Together with the work on polarity and volatility of bioactive substances as described above, the use of liquid chromatography coupled to high-resolution mass spectrometry has become the predominant technique used for the identification and structural elucidation of active substances. Data collected using this instrument allow for the fingerprinting of samples and fractions. In combination with Mass Profiler Professional (MPP) software, molecular composition differences among samples were studied using both reversed phase separation (C18) and normal phase separation (HILIC). FIGS. 6A, 6B, and 6C show the SEC separation of Amish dust samples (FIG. 6A) and subsequent analysis using LC-QTOF for both C18 (FIG. 6B) and HILIC (FIG. 6C) columns. Molecular feature extraction for the two separations still shows a complex matrix with potentially hundreds of components present.

SEC yields a limited number of highly active fractions: The bioassay-directed screening of protective substances contained in fractionated Amish extracts is shown in FIG. 4B. Aqueous extracts (100 mg/ml of dust equivalent) were fractionated by SEC, collecting fractions every 1.5 minutes, for a total of 18 fractions. After mass adjustment to the original concentration (100 mg/ml of dust equivalents), the recovered 18 fractions were used to stimulate 16HBE14o− human airway epithelial cells. Much of the protective bioactivity (TEER upregulation) was contained in DB fraction; (DA-DD, molecular weight: 28-51.5 kDa).

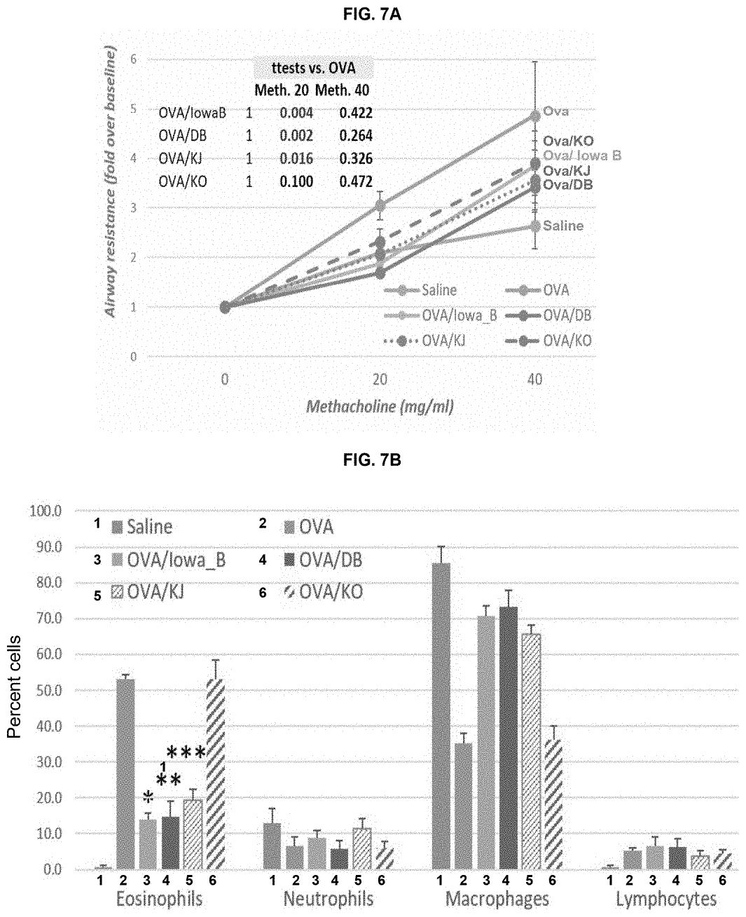

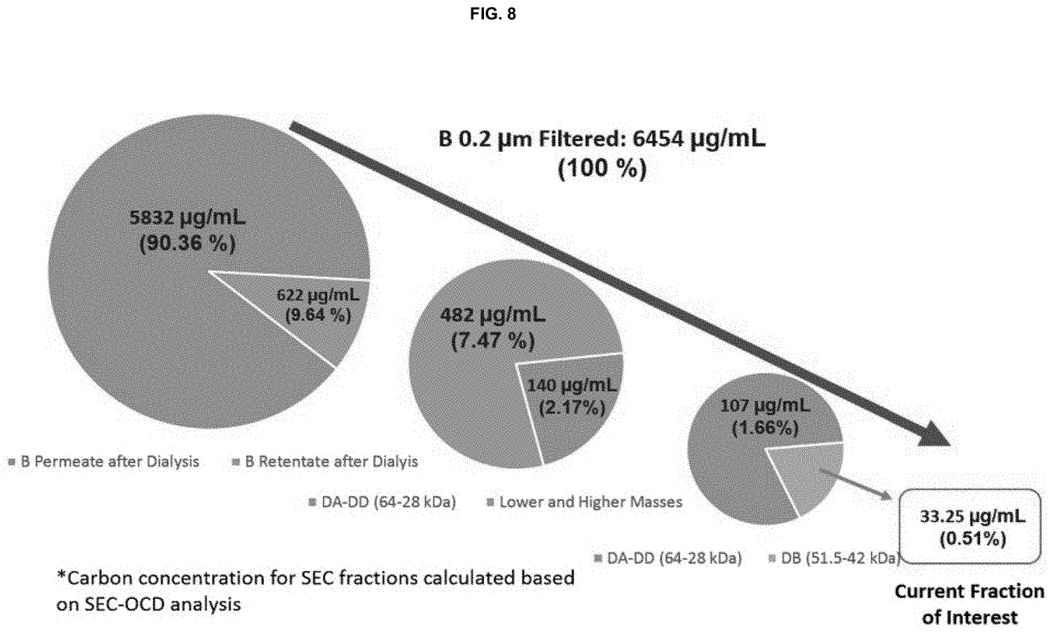

In vitro and in vivo activity of fraction DB: An abbreviated in vivo model of OVA-induced AHR and lung eosinophilia is shown in FIG. 2B and was used to repeatedly assess asthma- and allergy-protective activity following intranasal administration of Amish extracts or those among their fractions that had exhibited activity in vitro. Initial focus was on SEC fraction DB (SEC eluate representing structures with molecular weights of ≈42-51.5 kDa), which was 99% active in the epithelial cell assay at a very low concentration (e.g., 0.33 mg/well). FIGS. 7A and 7B show that fraction DB inhibited OVA-induced AHR and BAL eosinophilia significantly and almost as effectively as the parent unfractionated extract (IowaB). It is also noteworthy that fraction DB represents no more than 0.5% of the total carbon content of an unfractionated Amish aqueous extract (FIG. 8). Thus, the inhibitory activity contained in the DB fraction has been concentrated at least ≈200-fold relative to the original extract.

Focus on glycan-containing molecules: Mass spectrometry and NMR work has indicated the presence of polysaccharide-based structures in active fractions of Amish dust samples. To further investigate the presence of glycan-type structures, two different lectin-based affinity separation methods were employed: wheat germ agglutinin (WGA) and concanavalin A (ConA). Lectins are carbohydrate binding proteins that are highly specific for saccharide moieties. ConA targets α-D-mannosyl and α-D-glucosyl residue motifs, while WGA targets N-acetyl-D-glucosamine and sialic acid motifs. These lectins were selected based on glycan structures observed in both mass spectrometry and NMR experiments conducted on unfractionated Amish dust extract and fraction DB.

In initial experiments, agarose-bound ConA and WGA resins were used to isolate glycoconjugates (glycoproteins, glycopeptides, peptidoglycans, glycolipids, glycosides and lipopolysaccharides) from the DB fraction. A fraction of DB previously depleted of endotoxins/lipopolysaccharides was also produced. Substances not binding to lectin resin were collected and separated from substances with affinity for WGA and ConA. Molecules with affinity to the resin were eluted from the resins with eluent appropriate for each resin, and concentrated for bioassay. Only WGA flowthrough was able to inhibit airway eosinophilia induced by Ova. Similarly, WGA flowthrough from DA fraction was also inhibiting airway eosinophilia (data not shown).

Being highly polar and endotoxin free, fractions still preserved proteolytic activity as measured by Fluorescein isothiocyanate (FITC)-Casein fluorescent protease assay and confirmed by cytokine degradation monitored by mass spectrometry and Enzyme-linked immunosorbent assay (ELISA). Presence of relevant carbohydrate/polysaccharide was also evaluated by a lectin array and monosaccharide analysis, showing not only the presence of NeuElisa5Gc, but also αMan, αGlc, (GlcNAc)2-4, Galβ3GalNAc, GalNAcα(1,3)[αFuc(1,2)Gal, Galβ4GlcNAcβ2Manα6(GlcNAcb4), GlcNAc concentration. (from top 7 lectin interactions with at least 0.2 normalized intensity relative to positive control). Arabinogalactan concentration as measured by radial diffusion in agarose gel with Yariv reagent, reached 3.3 mg/mL in the raw extract but could not be detected in final active preparations.

### In Vitro and In Vivo Activity of Fractions DA-DE:

TABLE 2
In Vivo Activity of Fractions DA-DE and Their
WGA Retentate/Flowthrough Subfractions.

| | | | | BAL |
|---|---|---|---|---|
| Experiment | | | AHR | eosinophilia |
| Number | Fraction | Specs | inhibition¹ | inhibition² |
| 98 | DA | parent | ND | Yes |
| 107 | DA | parent | No | Yes |
| | KI | WGA FT³ | No | No |
| | KN | WGA RET⁴ | No | No |
| 83 | DB | parent | Yes | No |
| 95 | DB | parent | No | Yes |
| 105 | DB | parent | Yes | Yes |
| | KJ | WGA FT | Yes | Yes |
| | KO | WGA RET | No | No |
| 96 | DC | parent | Yes | No |
| 106 | DC | parent | Yes | No |
| | KK | WGA FT | Yes | No |
| | KP | WGA RET | No | Yes |
| 98 | DD | parent | ND | Yes |
| 109 | DD | parent | Yes | Yes |
| | KL | WGA FT | Yes | No |
| | KQ | WGA RET | No | No |
| 98 | DE | parent | ND | No |
| 110 | DE | parent | No | No |
| | DM | WGA FT | No | No |
| | DR | WGA RET | No | No |

¹Significant (p < 0.05) decrease in AHR for at least one methacholine dose,
²Significant (p < 0.05) decrease in BAL eosinophilia,
³FT = flow-through,
⁴RET = retentate

The spectrum of protective activities contained in all the fractions found to be active in the airway epithelial cell assay (DA, DB, DC and DD) as well as DE (negative control), both as such and as WGA flowthrough/retentate subfractions was repeatedly assessed. The in vivo model shown in FIG. 2B was used, and a summary of the results obtained for all fractions is shown in Table 2. Some variability is observed when individual fractions and subfractions are repeatedly tested in vivo. This variability might reflect the need for further standardization of fraction preparation methods (different experiments were performed with different preparations of the “same” fraction). Also, since the mass in these samples represents a minute proportion of the mass present in unfractionated Amish dust, dust consumption was limited by not mass-adjusting the WGA preparations. However, even under these conditions several of these preparations were active in vivo, i.e., they significantly inhibited AHR (WGA flowthrough of fractions DB, DC and DD) and/or BAL eosinophilia (WGA flowthrough of fractions DA, DB and DD).

Identification of glycosylated proteins present in DB: Information regarding protein concentration and carbohydrate content of DB fractions suggests that glycoproteins represent a large portion of the molecules present in the extract. In order to identify core protein structures, both in-solution and in-gel SDS-PAGE enzymatic digestion/peptide mapping approaches were applied and led to the identification of eight structures (Table 3). Notable among them were odorant-binding protein and the allergen Bos d2, both lipocalin transport proteins of bovine origin. As lipocalins, odorant binding protein and Bos d 2 have biological and immunological properties that warrant further investigation of their in vivo asthma/allergy protective role. These proteins were present both as retained and non-retained to WGA lectin affinity columns, indicating that the glycosylation patterns attached to these proteins have significantly different structural orientations. Identifications were completed using in gel digestion followed by peptide mapping using data dependent acquisition by an Agilent 6450 Q-TOF instrument. Data extraction and peptide/protein searches were conducted using Spectrum Mill MS Proteomics Workbench software (Agilent) with comparison to the SwissProt database.

For further protein structural conformations of DA, DB, and DC fractions, proteins were separated by SDS-PAGE and digested to reveal amino acid sequence coverage using LC-QTOF. These structures represent the only protein structures identified in both retained and flow-through WGA fractions of DB. No proteins were identified which could be attributed to the WGA resin itself. Moreover, several of these structures have some reported immunological activity. For instance, the allergen Bos d2 has been reported to have immunomodulatory activity. Odorant-binding protein is abundantly expressed in the nose. Both proteins are bovine lipocalins, soluble carrier proteins with functional properties that depend on the load of their pocket. Provicilin and vicilin are major allergens from peas. Conglycinin is a soybean storage protein that also has strong allergenic properties. Interferon-γ is a pleiotropic cytokine.

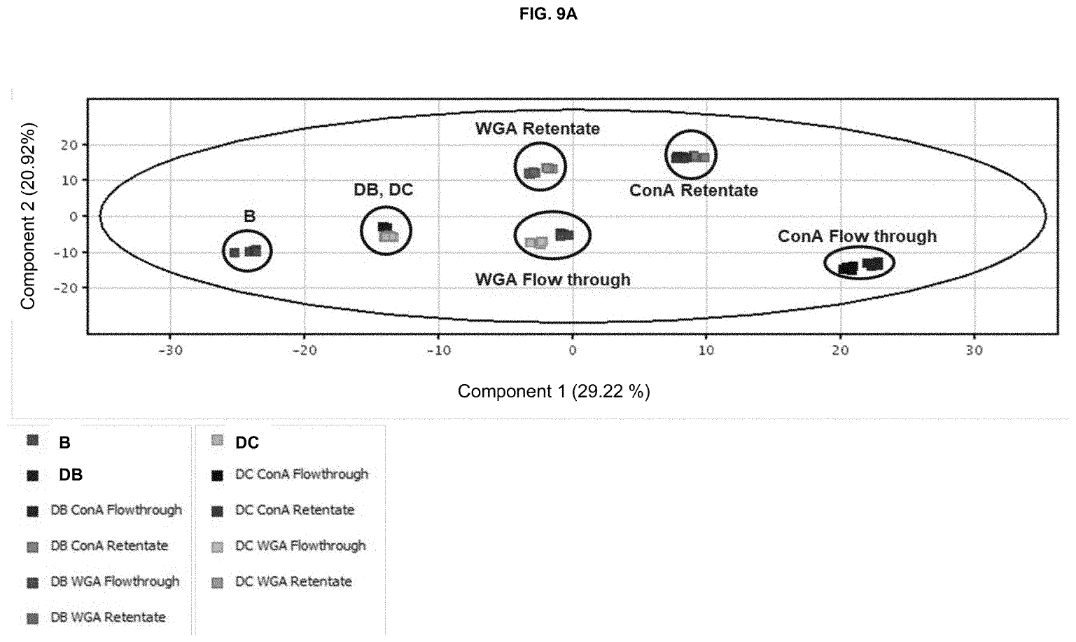

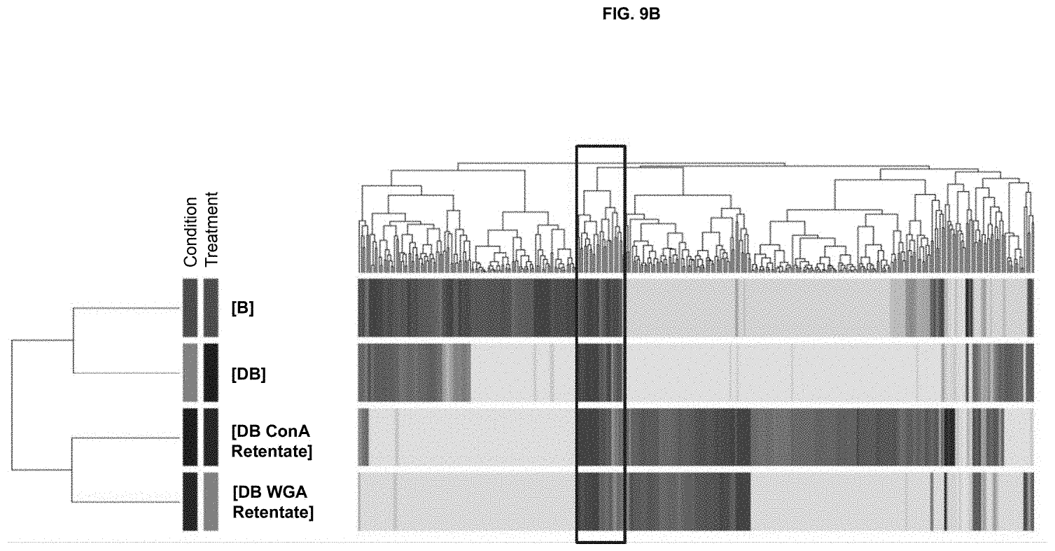

FIGS. 9A and 9B show glycan-containing molecules mass spectrometry analysis. FIG. 9A shows Principal Components Analysis (PCA) of raw sample, SEC fractions and lecithin-treated samples. FIG. 9B shows Hierarchical Clustering Analysis (HCA) of Entities and Sample Treatments for relevant glycan-containing samples.

TABLE 3
Initial Identification of Target Protein Structures
Present in Active Amish Dust Extracts (fraction DB).

| | Database | Protein | |
|---|---|---|---|
| # | Accession # | Name | Organism |
| 1 | P07435 | Odorant-binding protein | Bos Taurus (Bovine) |
| 2 | Q28133 | Allergen Bos d 2 | Bos Taurus (Bovine) |
| 3 | P42161 | Interferon gamma | Canis lupus familiaris |
| | | | (Dog) |
| 4 | P02854 | Provicilin | Pisum sativum |
| | | | (Garden pea) |
| 5 | P02856 | Vicilin, 14 kDa Comp. | Pisum sativum |
| | | | (Garden pea) |
| 6 | P13916 | Beta-conglycinin | Glycine max |
| | | | (Soybean) |
| 7 | P86909 | Sarcoplasmic calcium- | Chinoecetes opilio |
| | | binding protein | (Crab beetle) |
| 8 | Q9M2H6 | MATH domain At3g58400 | Arabidopsis thaliana |
| | | | (Mouse ear cress) |

Bulk characterization of bioactive extracts: Characterization and standardization of naturally derived complex preparations is challenging due to the inherent diversity of structures present. Total organic content (TOC), total protein measured by BCA method, and carbohydrate estimated from sodium meta-periodate reaction were used as bulk extract parameters for sample characterization and standardization. Bulk parameters were also used to track extract complexity deconvolution. TOC reduction achieved from initial was 99.56% (from 6454 μg/mL to 28.7 μg/mL for a molecular weight defined and endotoxin free preparation). Similarly, total protein reduction achieved was 98.66% and total carbohydrate was 99.2%, further attempts to deconvolute this preparation lead to biologically inactive preparations (data not shown). Final endotoxin concentration was less than 0.1 EU/mL.

FIGS. 10A and 10B show fraction DC and endotoxin-depleted DC, but not the endotoxin column eluate, inhibit both AHR and BAL eosinophilia. Activity of these preparations after intranasal administration was tested in the in vivo model presented in FIG. 2B. Measurements of airway resistance in response to methacholine challenge and total BAL cellularity are shown in the left and right panel, respectively. Statistical differences were assessed by Student's t test.

FIG. 11 shows Inhibitory activity of fractions DA, DD and DE on OVA-induced BAL eosinophilia. The activity of these preparations after intranasal administration was tested in the in vivo model presented in FIG. 2B. BAL cellularity is shown. Statistical differences were assessed by Student's t test. *p=0.0003; *p=0.001; ***p=0.01.

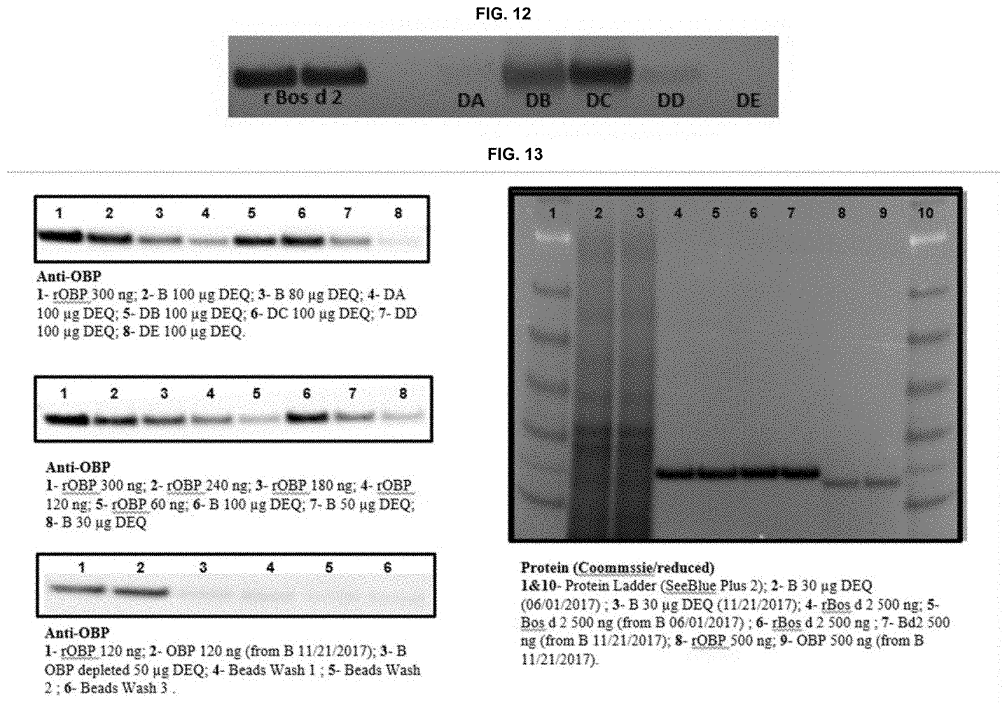

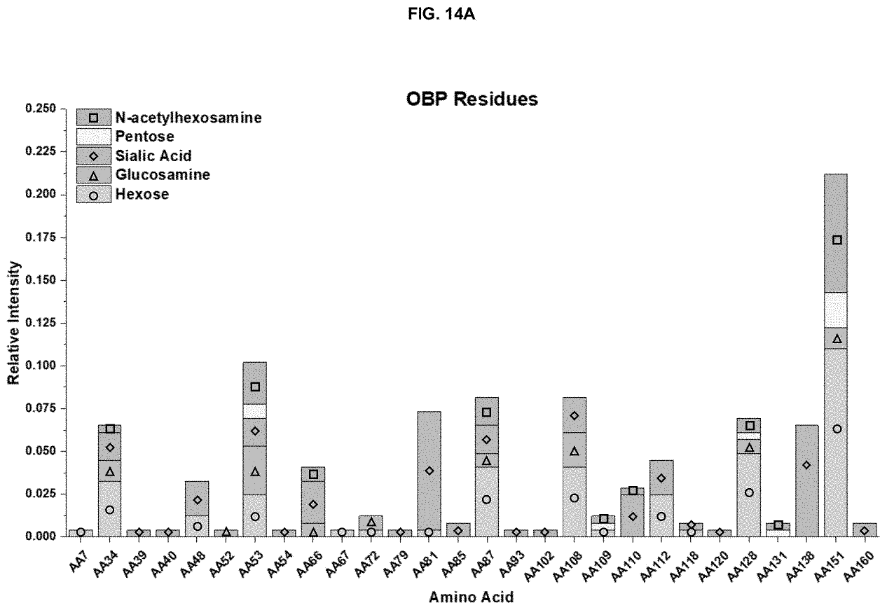

Immunoprecipitation and Quantities of proteins: The quantities of bovine Bos d2 (FIG. 12) and OBP (FIG. 13) contained in Amish dust extracts and their different fractions were determined by SDS-PAGE followed by Western blot or SDS-PAGE only (for purified proteins) using recombinant proteins as standards. FIG. 12 shows analysis of Bos d2 in recombinant form or isolated from Amish dust extract fractions DA-DE. Loading: rBos d2: 62.5 ng/lane, fractions: 30 mg of dust equivalent/lane. Calculations: 5 mg of dust equivalents of DC contains ˜10.4 μg of Bos d2; 5 mg of dust equivalents of DB ˜2-5 μg of Bos d2. ˜120 ng of OBP in 50 μg of DEQ of B extract->12-15 μg/5 mg of dust equivalents (DB and DC contain similar amounts).

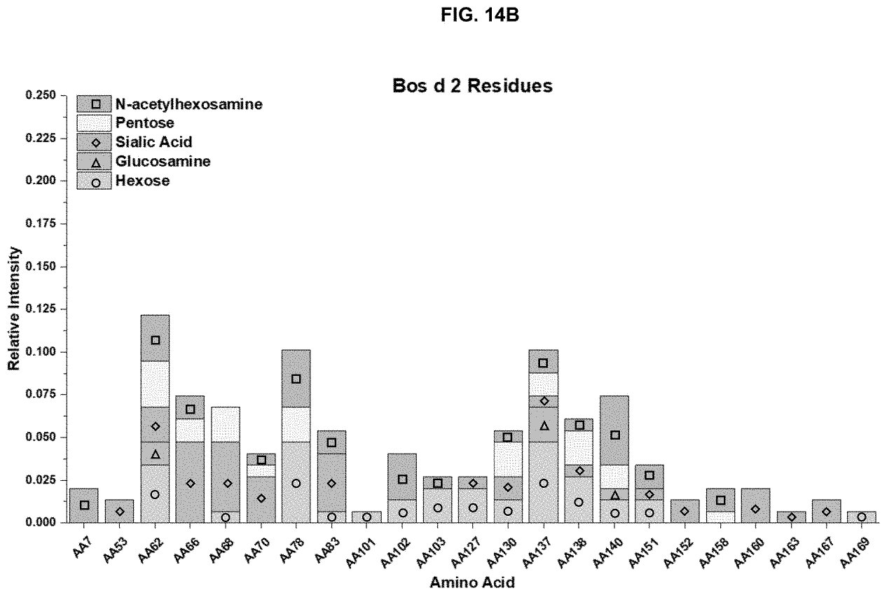

Bos d2 and OBP confirmation, glycosylation patterns and transported molecules identities of immunoprecipitated proteins: Bos d2 and OBP confirmation, glycosylation patterns and transported molecules identities of immunoprecipitated proteins were confirmed by enzymatic digestion/peptide mapping. Sequence coverage was determined by BioConfirm Software; for both protein isolates sequence coverage achieved was 100%. Using a similar strategy of mass spectrometry and BioConfirm software, glycosylation distribution for both proteins was determined (FIGS. 14A and 14B). For both samples, a predominance of hexose residues was found, followed by sialic acid residues for OBP and N-acetylhexosamine residues for Bos d 2. Glycosylation patterns found were similar to those predicted by in silico prediction tools (GlycoEP 1.0 N, Glyco EP 1.0 O, NetGlycate 1.0, NetNGlyc, and GPP). For identification of transported molecules stored inside isolated proteins, an untargeted liquid chromatography-high resolution mass spectrometry approach was used based on small molecules analysis parameters. Both positive and negative mode ionization experiments were conducted. Molecules identified consisted of a mixture of polyunsaturated fatty acids (PUFAs). Table 4 lists the structures identified for both samples. Similar structures were observed for both proteins, with 7 molecules identified in both samples.

TABLE 4
Identified Transported Structures for Bos d2 and OBP.

| Bos d2 | OBP |
|---|---|
| 11-HpOME | 11-HpOME |
| 9,12-dihydroxy stearic acid | 9,12-dihydroxy stearic acid |
| 12-oxo-10E-octadecenoic acid | 12-oxo-10E-octadecenoic acid |
| 9-hydroperoxy-10E,12,15Z- | 9-hydroperoxy-10E,12,15Z- |
| octadecatrienoic acid | octadecatrienoic acid |
| 9,10,13-trihydroxy-11-octadece- | 9,10,13-trihydroxy-11-octadece- |
| noic acid | noic acid |
| 9-hydroxy-10-oxo-12-octadecenoic | 9-hydroxy-10-oxo-12-octadecenoic |
| acid | acid |
| 9R,10S,18-trihydroxy-stearic acid | 9R,10S,18-trihydroxy-stearic acid |
| α-12(13)-EpODE | 6-ethyl-tetradecanoic acid |
| 3-keto palmitic acid | 11,15-dimethyl-hexadecanoic acid |
| 6E,8E,14E-Hexadecatriene-10,12- | 11-hydroperoxy-12,13-epoxy-9- |
| diynoic acid | octadecenoic acid |
| 9,12-Octadecadiynoic acid | |
| 8-Octadecenoic acid, 10-hydroxy- | |
| 12-oxo,13-hydroxy-9Z-octa- | |
| decenoic acid | |

In summary, several in vitro and in vivo screening assays were developed to monitor the activity of farm dust extracts and fractions thereof generated through a variety of procedures used to monitor the ability of Amish farm dust extracts and their fractions to (a) upregulate trans-epithelial electrical resistance (TEER, a critical component of mucosal barrier integrity) when added in vitro to serum-starved 16HBE14o− human airway epithelial cells, and (b) induce γδT (gdT) cells in vivo in the mouse lung when inhaled 4-5 times over an 8-10 day period. After screening dozens of Amish dust extract fractions, a subset of biologically active fractions was identified that were then validated in vivo using robust experimental asthma protocols.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A barn dust composition with asthma-protective properties, the barn dust composition comprising one or more bioactive fractions extracted from barn dust, said one or more bioactive fractions comprising one or more proteins and one or more fatty acids.

2. The composition of claim 1, wherein the one or more proteins comprise one or more transport proteins, wherein the one or more transport proteins are selected from a group consisting of Bos d2, odorant-binding protein, interferon gamma, provicilin, vicilin (14 kDa component), beta-conglycinin, sarcoplasmic calcium-binding protein, and MATH domain At3g58400.

3. The composition of claim 2, wherein the one or more transport proteins carry the one or more fatty acids.

4. The composition of claim 1, wherein the one or more fatty acids comprises one or more polyunsaturated fatty acids (PUFAs), wherein the PUFAs are selected from a group consisting of 11-HpOME, 9,12-dihydroxy stearic acid, 12-oxo-10E-octadecenoic acid, 9-hydroperoxy-10E,12,15Z-octadecatrienoic acid, 9,10,13-trihydroxy-11-octadecenoic acid, 9-hydroxy-10-oxo-12-octadecenoic acid, 9R,10S,18-trihydroxy-stearic acid, α-12(13)-EpODE, 3-keto palmitic acid, 6E,8E,14E-Hexadecatriene-10,12-diynoic acid, 9,12-Octadecadiynoic acid, 8-Octadecenoic acid, 10-hydroxy-12-oxo,13-hydroxy-9Z-octadecenoic acid, 6-ethyl-tetradecanoic acid, 11,15-dimethyl-hexadecanoic acid, and 11-hydroperoxy-12,13-epoxy-9-octadecenoic acid.

5. The composition of claim 1, wherein the asthma-protective properties comprise preventing, suppressing, and/or abrogating airway hyperresponsiveness (AHR) and/or broncho-alveolar lavage (BAL) eosinophilia, and/or reducing IL-13, IL-4, IL-5, and/or IgE levels due to allergens, wherein the allergens comprise allergenic proteins, ovalbumin, house dust mites, cockroaches, Alternaria, pollen, farm dust, pet dander, and/or other environmental-, animal-, and/or plant-based allergens.

6. The composition of claim 1, wherein the one or more bioactive fractions comprise an unfractionated barn dust extract.

7. The composition of claim 1, wherein the barn dust comprises Amish farm barn dust or Hutterite farm barn dust.

8. The composition of claim 1, wherein the barn dust composition is a solubilized material.

9. The composition of claim 1, wherein the barn dust composition comprises less than 2.0% total organic carbon content, less than 0.2% endotoxins, less than 0.2% lipopolysaccharides, or a combination thereof as compared to an original barn dust sample.

10. The composition of claim 1, wherein the barn dust composition has proteolytic activity.

11. The composition of claim 1, wherein the barn dust composition is a pharmaceutical composition, wherein the pharmaceutical composition is administered orally, conjunctivally, subcutaneously, intraarticularly, intraperitoneally, rectally, or vaginally, or via nasal inhalation.

12. The composition of claim 1, wherein the barn dust composition is used to treat or prevent allergies comprising: allergic asthma, allergic rhinitis, allergic dermatitis, food allergies.

13. A method of preventing or treating allergies or asthma in a subject in need thereof, the method comprising administering a therapeutically effective amount of a barn dust composition comprising one or more bioactive fractions extracted from barn dust, said one or more bioactive fractions comprising one or more proteins and one or more fatty acids, to a subject.

14. The method of claim 13, wherein the one or more proteins comprises one or more transport proteins, wherein the one or more transport proteins are selected from a group consisting of Bos d2, odorant-binding protein, interferon gamma, provicilin, vicilin (14 kDa component), beta-conglycinin, sarcoplasmic calcium-binding protein, and MATH domain At3g58400.

15. The method of claim 13, wherein the one or more fatty acids comprises one or more polyunsaturated fatty acids (PUFAs), wherein the PUFAs are selected from a group consisting of 11-HpOME, 9,12-dihydroxy stearic acid, 12-oxo-10E-octadecenoic acid, 9-hydroperoxy-10E,12,15Z-octadecatrienoic acid, 9,10,13-trihydroxy-11-octadecenoic acid, 9-hydroxy-10-oxo-12-octadecenoic acid, 9R,10S,18-trihydroxy-stearic acid, α-12(13)-EpODE, 3-keto palmitic acid, 6E,8E,14E-Hexadecatriene-10,12-diynoic acid, 9,12-Octadecadiynoic acid, 8-Octadecenoic acid, 10-hydroxy-12-oxo,13-hydroxy-9Z-octadecenoic acid, 6-ethyl-tetradecanoic acid, 11,15-dimethyl-hexadecanoic acid, 11-hydroperoxy-12,13-epoxy-9-octadecenoic acid.

16. The method of claim 13, wherein the allergies comprise soy allergies, peanut allergies, pollens, allergic asthma, allergic rhinitis, allergic dermatitis, food allergies, or a combination thereof.

17. The method of claim 13, wherein the method reduces occurrence of asthma attacks or allergic reactions.

18. The method of claim 13, wherein the method treats the reaction to asthma triggers comprising physical exertion, allergies, and/or allergens.

19. An in vitro method to screen compounds in barn dust extracts, barn dust fractions or barn dust sub-fractions thereof for airway protectiveness using 16HBE14o− epithelial cells, the method comprising:
a) obtaining differentiated, confluent 16HBE14o− cells cultured in trans-wells plates;
b) stressing a portion of the wells with the differentiated, confluent 16HBE14o− cells by culturing them in a serum-free medium;
c) exposing the non-stressed and stressed cells to barn dust extracts, barn dust fractions or barn dust sub-fractions;
d) measuring trans-epithelial electrical resistance (TEER) after a period of time;
e) expressing results as % activity, with 100% activity corresponding to complete inhibition of the loss of TEER observed in epithelial cells cultured in serum-free medium alone;
wherein the barn dust extracts, barn dust fractions or barn dust sub-fractions are protective of airways when ≥50% activity in the TEER assay is observed.

20. The method of claim 19, wherein a period of time comprises 3, 12, 24, 48 or 72 hours.
